(19) United States
(12) Patent Application Publication
(10) Pub. No.: US 2026/0110682 A1
(43) Pub. Date: Apr. 23, 2026

(54) COMPOSITIONS AND METHODS TARGETING SWIP-10 AND MBLAC1 FOR THE THERAPEUTIC MODULATION OF COPPER DYSHOMEOSTASIS

(71) Applicant: Florida Atlantic University Board of Trustees, Boca Raton, FL (US)

(72) Inventors: Randy D. Blakely, Palm Beach Gardens, FL (US); Maureen K. Hahn, Jupiter, FL (US); Peter Rodriguez, Jr., Jupiter, FL (US)

(21) Appl. No.: 19/114,209

(22) PCT Filed: Sep. 22, 2023
(86) PCT No.: PCT/US23/33510

§ 371 Date: Mar. 21, 2025

Related U.S. Application Data
(60) Provisional application No. 63/376,993, filed on Sep. 23, 2022.

(51) Int. Cl. G01N 33/50; A61K 49/00

(52) U.S. Cl. CPC G01N 33/5091; A61K 49/0008; G01N 33/5038; G01N 33/5085; G01N 33/5088

(57) ABSTRACT

Methods for identifying agents or manipulations that modulate copper (Cu) dyshomeostasis in an Mblac1- or swip-10-dependent manner, and for identifying agents that support Cu-dependent neuronal health in a subject are provided.

### CROSS-REFERENCE TO RELATED APPLICATION

The present application claims priority to U.S. Provisional Application Ser. No. 63/376,993, filed Sep. 23, 2022, the entire disclosure of which is incorporated herein by reference in its entirety.

### FIELD OF THE INVENTION

The invention relates generally to the fields of medicine, neurology, pharmacology, and molecular biology. In particular, the invention relates to methods to identify agents or manipulations that modulate copper (Cu) dyshomeostasis in an MBLAC1- or SWIP-10-dependent manner in a subject and treating disorders associated with Cu dyshomeostasis.

### BACKGROUND

Proper copper (Cu) homeostasis, here referring to Cu intake, cellular, intracellular and systemic transport and elimination as well as the interconversion of Cu oxidation states (e.g. Cu++ (cupric)→Cu+ (cuprous)), is essential to the function and health of cells in microorganisms as well as humans. Cu+ is an essential micronutrient, participating in many biosynthetic pathways and playing a key role in supporting the ability of mitochondria to synthesize ATP and to buffer oxidative stress. Multiple human disorders of genetic and non-genetic origin feature disrupted Cu homeostasis, including, as examples, neurodegenerative disorders such as Alzheimer's disease ((AD) (Sensi et al. Trends Pharmacol Sci. 2018; 39:1049-1063)), amyotrophic lateral sclerosis (ALS), and Parkinson's disease (PD) (Acevedo et al., J Biol Inorg Chem vol 24(8):1141-1157, 2019)). A direct contribution of altered Cu homeostasis to neurodegeneration is evident in the CNS changes induced in Menkes and Wilson's diseases (Waggoner et al. Neurobiol Dis. 1999; 6:221-30) which involve mutations in Cu transport genes. The ability of Cu to modulate signaling receptors in the brain such as NMDA and GABA receptors (Gaier et al., J Neurotic Res. 2013 vol. 91(1):2-19), two widely distributed signaling molecules, indicates that altered Cu homeostasis is also likely to impact a wide spectrum of neurobehavioral disorders across the lifespan, not just neurodegenerative conditions.

The cholinergic hypothesis of AD posits deterioration of acetylcholine (ACh)-secreting neurons as a major cause of the decline in cognitive function in AD (Bartus et al. Science. 1982;217:408-417). As a result, acetylcholinesterase (AChE) inhibitors have been employed since the 1980s to prevent the synaptic degradation of ACh and enhance cholinergic neurotransmission. To date, three AChE inhibitors, donezepil, galantamine, and rivastigmine, and a combination of donepezil and memantine, an NMDA receptor antagonist, constitute the currently approved symptomatic medications for AD in the U.S, with only moderate efficacy, substantial dose-limiting side-effects, and no effect on underlying neuropathology (Sharma et al. Mol Med Rep. 2019;20:1479-1487). Without the development of effective therapeutics, millions of aging Americans face a future with the ravages of AD and other forms of neurodegenerative disorders. Many other disorders that have been linked to Cu dyshomeostasis remain poorly treated and would benefit from the development of effective therapeutics.

### SUMMARY OF THE INVENTION

The methods described herein are based on Cu dyshomeostasis induced by reduction or loss of SWIP-10/MBLAC1 protein and the use of cellular or animal models with altered SWIP-10/MBLAC1 to identify agents or manipulations that modulate Cu dyshomeostasis (e.g., that alleviate Cu dyshomeostasis such that Cu homeostasis is normalized in the subject) in an MBLAC1- or SWIP-10-dependent manner. It was discovered that loss of function mutations in the C. elegans gene swip-10 induces a reduction in cellular mitochondrial respiration, increased oxidative stress and neuronal degeneration. Aspects of the phenotype of swip-10 mutants can also be seen in cell and tissue preparations derived from a KO of the mouse swip-10 ortholog Mblac1. MBLAC1 protein has been shown to be an RNA endonuclease, involved in the synthesis and nuclear export of histone RNAs. One of these modified RNAs encodes histone H3 protein, with KD of Mblac1 expression in cell culture leading to reduced levels of histone H3 protein and changes in cell cycle, functions traditionally subserved by cell replication-dependent (RD) histone proteins. Importantly, H3 proteins have also been found to encode a Cu reductase (Cu2+→Cu+) function, independent of the role of H3 histones in nucleosome structure, DNA packaging into chromatin, and DNA replication. Importantly, mutations that disrupt Cu reductase function of H3 lead to increased oxidative stress in yeast, as observed in swip-10 mutant worms, and in cultured cells derived from Mblac1 knockout (KO) mice. The data described in the Examples below show that deficits in Mblac1swip-10 expression lead to a loss of Cu+ along with changes in the expression of genes and other biochemical features typically seen associated with oxidative stress. These changes are consistent with deficits in Cu+ homeostasis that ultimately lead to neuronal degeneration in worms and risk for neurodegenerative disease in humans. Consistent with this idea, a reduction in MBLAC1 mRNA expression has been identified in the brains of subjects with AD, and genetic markers linked to the Mblac11 gene have been found to be associated with AD that also demonstrates comorbid cardiovascular disease (AD-CVD) (Broce et al. Acta Neuropathol. 2019 Feb; 137(2):209-226). The data described herein support the model that reductions in SWIP-10/MBLAC1 protein levels compromise H3 histone mRNA processing, leading to diminished Cu+ availability. These changes in turn lead to reduced mitochondrial respiration, increased oxidative stress, and the altered health or death of neurons, as well as other disorders linked to Cu dyshomeostasis. Glial cells in the brain are major regulators of Cu homeostasis in the brain. Prior work has shown that the neurodegenerative consequences of deletion of the swip-10 gene derive from a loss of its expression in glial cells. The data described herein show that reduced mitochondrial function, elevated expression of genes linked to metabolic and oxidative stress, and neurodegeneration can be suppressed by selective glial expression of the wild type swip-10 gene as well by supplementation with Cu+. Whereas swip-10 is most highly expressed in C. elegans glia, the Mblac1 gene is expressed broadly in both the brain and periphery and rodents and humans, supporting a model whereby reductions in or loss of MBLAC1 protein leads to one or more peripheral disorders that may be comorbid with neurodegenerative or neurobehavioral disorders such as AD-CVD. In support of this model, from experiments described herein, changes in serum metabolites were identified. Additional experimental data demonstrated that the liver, the body's major organ for systemic Cu homeostasis, demonstrates altered metabolic function, consistent with changes in energy production (see, B. Ceyhan et al., “Optical imaging reveals liver metabolic perturbations in Mblac1 knockout mice” Engineering in Medicine and Biology Conference, 2023). Novel methods that target SWIP-10/MBLAC1, their regulators and substrates, for identifying therapeutics to remediate Cu dyshomeostasis and treat disorders with metabolic deficits and/or oxidative stress, including many functional and neurodegenerative disorders (such as AD, amyotrophic lateral sclerosis, and PD) and other disorders linked to Cu dishomeostatis, are described herein. Use of the reagents and tools discovered to diagnose and treat a human disease is also described herein.

Accordingly, described herein are methods of identifying agents or manipulations that modulate Cu dyshomeostasis in an Mblac1- or swip-10-dependent manner. The methods include: (i) providing a plurality of Mblac1 or swip-10 knockdown (KD) or KO animals, and a plurality of Mblac1 or swip-10 wild-type (WT) animals; (ii)exposing a first portion of the plurality of Mblac1 or swip-10 KD or KO animals and a first portion of the plurality of the Mblac1 or swip-10 WT animals to at least one test agent or manipulation, and exposing a second portion of the plurality of Mblac1 or swip-10 KD or KO animals and a second portion of the Mblac1 or swip-10 WT animals to a control vehicle; (iii) collecting a sample from each animal; (iv) measuring a value for at least one Cu marker in the samples from all the animals and comparing the effects of the at least one test agent or manipulation between the first portion of the plurality of Mblac1 or swip-10 KD or KO animals and the second portion of the plurality of the Mblac1 or swip-10 WT animals; and (v) identifying the at least one test agent or manipulation as one that modulates Cu dyshomeostasis if any of the following are observed: the value for the at least one Cu marker from the first portion of the plurality of Mblac1 or swip-10 KD or KO animals is statistically different from the value for the at least one Cu marker from the second portion of the plurality of Mblac1 or swip-10 KD or KO animals; the value for the at least one Cu marker from the first portion of the plurality of Mblac1 or swip-10 WT animals is statistically different from the value for the at least one Cu marker from the second portion of the plurality of Mblac1 or swip-10 WT animals; and the value for the at least one Cu marker from the first portion of the plurality of Mblac1 or swip-10 KD or KO animals is equal or similar to the value for the at least one Cu marker from the first and/or second portions of the plurality of the Mblac1 or swip-10 WT animals.

In the methods, modulating Cu dyshomeostasis includes alleviating Cu dyshomeostasis such that Cu homeostasis is normalized. In embodiments, the at least one Cu marker is selected from the group consisting of: one or more Cu redux states, a Cu-dependent enzyme, a Cu-binding protein, a Cu-dependent physiological response or behavior, a Cu-dependent process, and a change in RNA, protein, posttranslational protein modification, or metabolite linked to Cu homeostasis. In embodiments of the methods, in step iv), the effects of the at least one test agent or manipulation include a change relative to pre-exposure in at least one of: one or more Cu redux states, a Cu-dependent enzyme, a Cu-binding protein, a Cu-dependent physiological response or behavior, a Cu-dependent process, and a change in RNA, protein, posttranslational protein modification, or metabolite linked to Cu homeostasis. In embodiments, modulating Cu dyshomeostasis includes increasing Cu dyshomeostasis. In embodiments, the at least one test agent is one or more of: small molecule, drug, nucleic acid, protein, peptide, nanoparticle, virus, viral vector, Cu chelator in a swip-10/Mblac1-dependent manner, Cu chaperone in a swip-10/MBLAC1-dependent manner, enzyme that binds Cu, protein that removes Cu, Cu transporter, agent that modifies enzymatic activity of Cu in a swip-10/Mblac1-dependent manner, and histone enzymatic modifying agent that acts in a swip-10/Mblac1-dependent manner. The at least one manipulation can be one or more of: a genetic manipulation; a transcriptome manipulation, a metabolome manipulation, and an environmental manipulation linked to Cu dyshomeostasis. Genetic manipulation includes manipulation of a subject's genome. Examples of environmental manipulation include mitochondrial and behavioral stress. In embodiments, in step iii) the sample is brain tissue, a peripheral tissue (e.g., liver), a bodily fluid, or feces. In embodiments, in the plurality of MBLAC1 or swip-10 KD or KO animals, Mblac1 expression is knocked down or knocked out selectively in glial cells.

Also described herein are methods of identifying agents or manipulations that modulate Cu dyshomeostasis in an Mblac1- or swip-10-dependent manner. The methods include: (i)providing a plurality of Mblac1 or swip-10 KD or KO animals, and a plurality of Mblac1 or swip-10 WT animals; (ii) measuring a baseline value for at least one Cu marker for each animal; (iii) exposing each animal to at least one test agent or manipulation, wherein each animal provides its own baseline value prior to exposure; (iv) at a first post-exposure time point, measuring at least one exposure response value for the at least one Cu marker for each animal; (v) optionally, at one or more of a second, third, fourth, fifth and sixth post-exposure time point, measuring an exposure response value for the at least one Cu marker for each animal; (vi) averaging the WT exposure response values compared to the WT baseline values, averaging the KO/KD exposure response values compared to the KO/KD baseline values, and then comparing the WT average to the KO or KD average, wherein if the WT average is different relative to the KO or KD average, the at least one test agent or manipulation modulates Cu dyshomeostasis.

In embodiments of these methods, step (v) is performed and a time course of the exposure response to the at least one test agent or manipulation is determined. The at least one test agent can be detectably labeled. In embodiments, in step (ii), measuring a baseline value for at least one Cu marker includes collecting a sample from the animals, in vivo imaging of a tissue in the animals, or measuring physiological behavior of all animals. In step (iv), measuring at least one exposure response value for the at least one Cu marker can include collecting a sample from the animals, in vivo imaging of a tissue in the animals, or measuring physiological behavior of all animals. In embodiments, in step (ii) measuring a baseline value for at least one Cu marker and in step (iv) measuring at least one exposure response value for the at least one Cu marker, blood from the animals is analyzed and the at least one Cu marker is measured and Cu-dependent behavior in the animals is analyzed and measured. The samples can include blood, serum, saliva, urine or feces. In embodiments, modulating Cu dyshomeostasis includes alleviating Cu dyshomeostasis such that Cu homeostasis is normalized in the subject. In embodiments, the at least one Cu marker is selected from the group consisting of: one or more Cu redux states, a Cu-dependent enzyme, a Cu-binding protein, a Cu-dependent physiological response or behavior, a Cu-dependent process, and a change in RNA, protein, posttranslational protein modification, or metabolite linked to Cu homeostasis. Modulating Cu dyshomeostasis includes increasing (worsening) Cu dyshomeostasis. In embodiments, the at least one test agent is one or more of: small molecule, drug, nucleic acid, protein, peptide, nanoparticle, virus, viral vector, Cu chelator in a swip-10/Mblac1-dependent manner, Cu chaperone in a swip-10/MBLAC1-dependent manner, enzyme that binds Cu, protein that removes Cu, Cu transporter, agent that modifies enzymatic activity of Cu in a swip-10/Mblac1-dependent manner, and histone enzymatic modifying agent that acts in a swip-10/Mblac1-dependent manner. The at least one manipulation can be one or more of: a genetic manipulation; a transcriptome manipulation, a metabolome manipulation, and an environmental manipulation linked to Cu dyshomeostasis. In embodiments, in the plurality of MBLAC1 or swip-10 KD or KO animals, Mblac1 expression is knocked down or knocked out selectively in glial cells.

Further described herein are methods of identifying agents that support Cu-dependent neuronal health in a subject. The methods include: (i) providing a plurality of Mblac1 or swip-10 KD or KO animals, and a plurality of Mblac1 or swip-10 WT animals; (ii) exposing a first portion of the plurality of Mblac1 or swip-10 KD or KO animals and a first portion of the plurality of Mblac1 or swip-10 WT animals to a neural toxin or other neural insult that results in pathology in a Cu-dependent manner, wherein a second portion of the Mblac1 or swip-10 KD or KO animals and a second portion of the Mblac1 or swip-10 WT animals are not exposed to the neural toxin or neural insult as controls; (iii) measuring the pathology in each exposed animal; (iv) administering a test agent to at least the first portion of the plurality of Mblac1 or swip-10 KD or KO animals and the first portion of the plurality of Mblac1 or swip-10 WT animals; (v) measuring the pathology in each animal that was administered the test agent; (vi) identifying the test agent as an agent that supports Cu-dependent neuronal health in a subject if the test agent reverses the pathology of an Mblac1 KO. In the methods, reversing the pathology of an Mblac1 KO typically restores Cu homeostasis.

In embodiments, measuring the pathology in the animals includes measuring visually, biochemically, physiologically or behaviorally a marker of neuronal damage in the animals. In embodiments, the identified agent supports Cu-dependent neuronal health in the presence of MBLAC1 but not in the absence of MBLAC1. In embodiments, in step (ii), the neural toxin or other neural insult induces oxidative stress and/or neural degeneration in the animals. In embodiments, the identified agent has neuroprotective activity when administered to a mammalian subject in need thereof. In embodiments, the plurality of Mblac1 or swip-10 KD or KO animals are swip-10 KD or KO C. elegans animals, and the plurality of Mblac1 or swip-10 WT animals are swip-10 WT. C. elegans animals. Pathology can be measured by measuring proteins that are stimulated during cell death progression, by measuring the number of neural cells, or by an anatomical assay that counts healthy parts of a cell including dendrites or cell bodies. In embodiments, in the plurality of MBLAC1 or swip-10 KD or KO animals, Mblac1 expression is knocked down or knocked out selectively in glial cells.

Still further described herein are methods of identifying agents or manipulations that modulate Cu dyshomeostasis in an Mblac1- or swip-10-dependent manner involving cultured cells. The methods include: (i) providing a first group of cultured cells derived from a plurality of Mblac1 or swip-10 KD or KO animals, and a second group of cultured cells derived from a plurality of Mblac1 or swip-10 WT animals; (ii) exposing a first portion of the first group of cultured cells and a first portion of the second group of cultured cells to at least one test agent or manipulation, and exposing a second portion of the first group of cultured cells and a second portion of the second group of cultured cells to a control vehicle; (iii) measuring a value for at least one Cu marker in each of the portions and comparing the effects of the at least one test agent or manipulation between the first portion of the first group of cultured cells and the second portion of the second group of cultured cells; and identifying the at least one test agent or manipulation as one that modulates Cu dyshomeostasis if at least one of the following is observed: the value for the at least one Cu marker from the first portion of the first group of cultured cells is statistically different from the value for the at least one Cu marker from the second portion of the first group of cultured cells; the value for the at least one Cu marker from the first portion of the second group of cultured cells is statistically different from the value for the at least one Cu marker from the second portion of the second group of cultured cells; and the value for the at least one Cu marker from the first portion of the first group of cultured cells animals is equal or similar to the value for the at least one Cu marker from the first and/or second portions of the second group of cultured cells. In embodiments, test agents can be added repeatedly and in the presence of drugs that block Cu related pathways (e.g. Cu chelators) to thereby test the Cu dependence of changes observed with loss of swip-10 and Mblac1.

Additionally described herein are methods of identifying agents or manipulations that modulate Cu dyshomeostasis in an Mblac1- or swip-10-dependent manner involving cultured cells. The methods include: (i) providing a first group of cultured cells derived from a plurality of Mblac1 or swip-10 KD or KO animals, and a second group of cultured cells derived from a plurality of Mblac1 or swip-10 WT animals; (ii) measuring a baseline value for at least one Cu marker in both groups of cultured cells; (iii) exposing both groups of cultured cells to at least one test agent or manipulation, wherein each cultured cell provides its own baseline value prior to exposure; (iv) at a first post-exposure time point, measuring at least one exposure response value for the at least one Cu marker for both groups of cultured cells; (v) optionally, at one or more of a second, third, fourth, fifth and sixth post-exposure time point, measuring an exposure response value for the at least one Cu marker for both groups of cultured cells; and (vi) averaging the second group of cultured cells' exposure response values compared to the second group of cultured cells' baseline values, averaging the first group of cultured cells' exposure response values compared to the first group of cultured cells' baseline values, and then comparing the second group of cultured cells' average to the first group of cultured cells' average, wherein if the second group of cultured cells' average is different relative to the first group of cultured cells' average, the at least one test agent or manipulation modulates Cu dyshomeostasis.

Yet further described herein are methods of administering a therapeutic agent(s) identified in the methods described herein to a subject in need of such treatment, i.e., a subject who has a disease or disorder associated with Cu dyshomeostasis. In such methods, a therapeutically effective amount of a therapeutic agent is administered to a subject (e.g., a human) having a disease or disorder caused by Cu dyshomeostasis. Typically, the therapeutically effective amount results in at least one of the following desirable results: induction or enhancement of mitochondrial respiration, enhancement (promotion) of neural cell health, reduction of neural cell death, suppression of oxidative stress, and prolonging of survival in the subject. A disease or disorder associated with Cu dyshomeostasis can be a disease or disorder that has been reported to exhibit, any of, as examples, oxidative stress, mitochondrial dysfunction, neural degeneration, etc. Examples of diseases associated with Cu dyshomeostasis include AD, PD, Menkes disease, Wilson disease, fatal infantile cardioencephalomyopathy, metabolic syndrome, anemia, cardiovascular disease, cancer, neurodegenerative disease, diabetes, etc.

### BRIEF DESCRIPTION OF THE DRAWINGS

FIG. 1A-1G show a model for how SWIP-10/MBLAC1 participate in Cu+ dependent regulation of mitochondrial energetics and suppression of oxidative stress. FIG. 1A: SWIP by analogy to MBLAC1 is a 3′ pre-mRNA endonuclease whose activity contributes to translation of RD histone protein. FIG. 1B: Genomic DNA tightly wrapped around histone proteins that form chromatin within the cell nucleus. FIG. 1C: RD histones assemble as nucleosomes and promote DNA compaction into chromatin. FIG. 1D: H3 histones encode a Cu++ reductase activity in the interior of the histone octamer that ensures adequate levels of Cu+ for cellular functions, FIG. 1E-1G specifically as critical cofactors for mitochondrial cytochrome oxidase (COX) comprising Complex IV of the electron transport chain and for superoxide dismutases such as SOD1 whose mutation constitutes the most common hereditary form of ALS.

FIG. 2A-2E show in vivo measurements of O₂ consumption rates decreased in swip-10 mutants that indicate the rate of mitochondrial respiration. FIGS. 2A and 2C: Experiments were conducted with WT (N2) and swip-10 KO worms using a Seahorse XF96 Respirometer and yielded a highly significant difference in basal O₂ consumption rates using Welch's two sample t-test. P<0.0003 (measurements 0-7). Treatment with FCCP (FIGS. 2A and 2D), a mitochondrial uncoupler, restores oxygen consumption in swip-10 KOs, indicating that loss of O₂ consumption lies in the dysfunction of the electron transport chain or in the generation of carbon substrates needed to support mitochondrial electron transport. Sodium azide treatment (FIGS. 2A and 2E) establishes the level of non-mitochondrial oxidative respiration, which is found to be equivalent in swip-10 KOs and WT worms. FIG. 2B: Independent validation of a loss of basal mitochondrial oxygen consumption in swip-10 KO worms using the Oroboros Oxygraph 2K respirometer (swip-20 is a different worm line, studied here for unrelated reasons). Data here are presented as mean oxygen consumption (pmol/second) of 100 worms. ** represents statistical significance (P<0.01) compared to the N2 strain by a two-tailed (unpaired) t-test.

FIG. 3A-3D show evidence of whole animal oxidative stress in swip-10 mutants. Nematodes (100/group) at the L4 stage were subjected to LC/MS-MS based measurements of reduced glutathione (GSH) (FIG. 3A), oxidized glutathione (GSSG) (FIG. 3B), and of the ratio of GSH/GSSG (FIG. 3C). Both swip-10 and swip-20 lines demonstrated diminished levels of GSH and elevated levels of GSSG, leading to an overall elevated ratio of GSH/GSSG. These findings mirror those observed in cultured cells prepared from Mblac1 KO mice (Samantha McGovern FAU Masters Thesis, 2019). In FIG. 3D, additional evidence of oxidative stress in swip-10 mutants via measurement of DCFDA, a molecule that increases in fluorescence in the presence of reactive oxygen species (ROS), is provided.

FIGS. 4A and 4B show gene expression changes in swip-10 mutant worms that support the induction of protective responses to metabolic/oxidative stress and altered Cu homeostasis. FIG. 4A: mRNA from WT (WT value=black horizontal line) and swip-10 mutant worms was extracted and subjected to qPCR to quantify changes in the expression of genes linked to metabolic/oxidative stress (skn-1, gst-4), to lactate and pyruvate efflux via glial monocarboxylate transporter (mct-1/2), and to the Cu+ transporter chca-1 or the Cu+ chaperone cuc-1. Significant elevations were found in swip-10 animals over WT for skn-1, gst-4, chca-1 and cuc-1, whereas mct-1 gene expression was significantly reduced. FIG. 4B: chca-1 RNA levels in swip-10 animals are elevated to a similar extent as in N2 worms treated with the Cu+ specific chelator bathocuproinedisulfonic acid (BCS), revealing that loss of swip-10 has as profound an effect on the expression of a Cu-dependent gene as does Cu+ chelation. skn-1 encodes a transcription factor and ortholog of mammalian Nrf2 that is responsive to oxidative stress, increasing expression of downstream neuroprotective genes. mct-1/2 encodes a transporter of lactate/pyruvate that are involved in bioenergetic pathways. chca-1 encodes a Cu+ transporter.

FIG. 5A-C show an elevation of Nrf2 protein levels in prefrontal cortex of Mblac1 KO vs WT littermates, consistent with elevated oxidative stress occurring in the absence of MBLAC1 protein. The Nrf2 gene is the mammalian ortholog of the oxidative stress-sensitive worm gene skn-1 (shown in FIG. 4A). Western blot analysis of prefrontal cortex (PFC, FIG. 5A) and hippocampus (Hipp, FIG. 5B), with bands quantified in FIG. 5C. After image capture (Licor), the blot was stripped and reprobed with antibody to beta actin. Levels of Nrf2 were normalized for actin levels in the same sample and then statistically analyzed by a two-tailed, unpaired Student's t-test. *=p<0.05, one-tailed Student's t-test.

FIGS. 6A and 6B show specific chelation of Cu+ induces neurodegeneration in WT worms whereas Cu+ supplementation rescues the degeneration seen in swip-10 animals. FIG. 6A: WT transgenic worms with GFP-labeled DA neurons (BY250) were raised on agar plates containing vehicle or 10 μM of the Cu+ specific chelator BCS and imaged by fluorescence microscopy at the late L4 stage to quantify DA neuron degeneration. Data derived from 4 experiments for each condition with statistical evaluation by a one-tailed Student's t-test. FIG. 6B: To demonstrate that loss of DA neurons in swip-10 mutants involves a loss of Cu+, WT (BY250) or BY250; swip-10 animals were incubated with or without CuCl2 (10 μM) or the organic Cu+ chaperone elesclomol (5 μM). CuCl₂ is known to generate Cu+ upon uptake into cells, thus elevating intracellular Cu+ levels. Data derived from 4 experiments for each condition, with data analyzed by one-way ANOVA and post-hoc tests versus BY250; swip-10. **=<0.01, ****=p<0.0001.

FIG. 7 shows quantitative detection of diminished Cu+ levels in swip-10 mutants vs WT worms using the Cu+ specific fluorophore CF4 as compared to a lack of observed changes using the Cu+ insensitive fluorophore CF4-CTL. WT or swip-10 mutants at the late LA state were incubated on plates containing 25 μM CF4 or CF4-CTL solution for 12-16 hrs and the number of fluorescence storage granules (puncta) are quantified by confocal microscopy. The CF4 dye forms fluorescent puncta when bound to Cu+ that is proportional to Cu+ content. Each point is an average from a single day where 5 animals were measured. swip-10 mutants had significantly reduced puncta compared to WT as determined by a two-tailed Student's t-test (*=p<05). No significant change was observed when comparing WT to swip-10 using the Cu+ insensitive dye CF4_CTL.

FIG. 8A-C show that reduced mitochondrial function, elevated gene expression, and oxidative stress are suppressed by expression of wild type swip-10 selectively in glial cells, using the pan glial promoter ptr-10. 8A: Basal OCR determined by Oroboros Oxygraph respirometer as an average of steady state recordings over 10 minutes. One-way ANOVA used for analysis. **p≤0.01. 8B: Relative gene expression levels quantified via qPCR. All gene expression values were normalized to the housekeeping gene actin. Data shown as 95% confidence intervals of the mean. Dashed line positioned at a value of 1 reflects WT levels of gene expression. 8C: Measurements of ROS levels of animals treated with the fluorescent ROS sensor DCFDA. Confocal microscopy images obtained were used to quantify relative fluorescence intensity normalized to size of each animal. Welch's t-test used for statistical analysis. *p≤0.05.

FIG. 9 is a graph showing the impact of loss of swip-10 on b-amyloid plaques produced in the GMC101 line. The GMC101 line expresses plaque forming human b-amyloid (1-42) peptide in muscle cells. The GMC101; swip-10 line expresses b-amyloid (1-42) in the context of a deleted swip-10 gene, modeling the increased risk seen in humans for AD-CVD when MBLAC1 gene expression is reduced. Plaques were stained with a fluorescent Congo-red derivative to allow for visualization and quantitation of b-amyloid plaques. Data were analyzed by a one-way ANOVA with post-hoc tests of significance at different ages of the worms, demonstrating significantly greater plaque deposition in the context of a swip-10 mutation. *=p<0.05, ***=p<0.001, ****=p<0.0001.

FIG. 10 is an analysis of serum biomarkers typically assayed to interrogate changes in organ function in mammals. Blood from 18-20 month old WT and Mblac1 KO mice was collected at sacrifice and spun to isolate serum. Serum samples were assayed commercially to evaluate biomarkers as follows: BUN=blood urea nitrogen, a measure of kidney function; cholesterol, produced systemically, predominantly by the liver; glucose, evaluated to examine liver and pancreatic function and sugar uptake/storage, as altered in metabolic syndromes; ALT=alanine transaminase, a measure of liver disease; CPK=creatine phosphokinase, a measure used to assess muscle and cardiac dysfunction; creatinine, a measure of kidney function; CRP=C-reactive protein, a measure of systemic inflammation that can be elevated in metabolic or infectious disease; Troponin I, a measure of cardiac muscle damage; Troponin II, a measure of cardiac muscle damage. Findings show a significant elevation in serum glucose, decreases in cholesterol levels, and elevated ALT and CPK levels, and reduced creatinine, suggesting a metabolic syndrome with likely involvement of liver, kidney and muscle.

### DETAILED DESCRIPTION OF THE INVENTION

Described herein are methods for identifying agents or manipulations that modulate Cu dyshomeostasis (e.g., restore Cu homeostasis) in an Mblac1- or swip-10-dependent manner utilizing Mblac1 or swip-10 KD or KO animals. Methods that utilize cultured cells derived from the Mblac1 or swip-10 KD or KO animals, as monolayers, suspension cultures, or in 3D culture (e.g. organoids), as well as cellular extracts therefrom, are also described herein. The swip-10 gene was identified in a screen for genes that normally constrain synaptic DA signaling. Genetic, pharmacological, optical and behavioral studies revealed that swip-10 is required in glia to limit DA neuron hyperexcitability arising from glutamate (Glu) receptor hyperactivation, triggering Swimming-Induced Paralysis (Swip). More recent studies revealed that the DA neurons in swip-10 animals display age-dependent DA neuron degeneration. As shown in the Examples below, swip-10 mutants show signs of global deficits in mitochondrial respiration and oxidative stress as well as neurodegeneration. Neurodegeneration in the context of Glu-dependent hyperexcitation and metabolic insufficiency/oxidative stress parallels mechanisms proposed for multiple neurodegenerative diseases including AD and PD. The human swip-10 ortholog MBLAC1, was recently identified in genome-wide association (GWAS) studies to be a risk gene for AD-CVD. MBLAC1 has been revealed to function as a RD histone pre-mRNA processing enzyme (Pettinati et al. eLife. 2018; 7:e39865). Recently, the RD histone protein H3 was found to be a (Cu) reductase (Cu2+→Cu+) independent of its role in chromatin remodeling and transcriptional regulation (Attar et al., Science. 2020;369:59-64). Cu+ is required for enzymes supporting both mitochondrial respiration and suppression of oxidative stress, whereas Cu²⁺ accelerates the aggregation of toxic proteins associated with both AD and PD. The role of MBLAC1 in regulating H3 histones that act as a Cu reductase adds further support to MBLAC1's role in mitochondrial function and oxidative stress. The data described herein suggest that swip-10/MBLAC1 are critical determinants of glial Cu+ homeostasis and are essential to glial support of neuronal excitability, function and viability. In the experiments described herein it was shown that treatment of swip-10 mutant worms with a Cu+ chaperone that boosts Cu levels suppresses neurodegeneration. It was also shown that mitochondrial respiration is affected in liver of Mblac1 KO animals (B. Ceyhan et al., “Optical imaging reveals liver metabolic perturbations in Mblac1 knockout mice” Engineering in Medicine and Biology Conference, 2023) with the Mblac1 KO animals exhibiting a loss of energetic cofactors and greater oxidized state compared to Mblac1 WT animals, consistent with whole body reductions in mitochondrial respiration consistent with whole body mitochondrial respiration deficits in swip-10 mutants.

### Knockdown (KD) and Knockout (KO) Animals

In the methods described herein, KD and/or KO animals are used. A KD animal is one in which the expression of one or more of the animal's genes is reduced. Transient, inducible, and reversible KD animals can be used. Methods of knocking down gene expression are well known in the art. See, e.g., Tiscornia et al. Proc Natl Acad Sci U S A. 2003 Feb. 18; 100(4): 1844-1848; Chang et al., Am J Pathol. 2004 Nov; 165(5): 1535-1541; Fire et al., Nature. 391 (6669): 806-11, 1998; U.S. Patent Applications pub. nos. 20100299771 and 20060277610, all of which are incorporated herein by reference. Customized KD rodents are commercially available from, e.g., Taconic Biosciences (Germantown, NY). Reduction of the function of a gene of interest, for example using molecules that regulate SWIP-10/MBLAC1 activity or their interactions with regulatory proteins, can also be used to establish a screening platform. KO animals have either a deletion or complete loss-of-function modification of the gene of interest. Lines of animals in which both copies of the gene (one on each chromosome) are knocked out in all tissues are referred to as homozygous KOs. Global and conditional KO rodent models can be made using, for example, CRISPR/Cas9 in either rodent zygotes or ES cells, on a chosen genetic background. Because the experiments described herein demonstrate rescue of phenotypes by expression of WT swip-10 in glial cells, because glia are a major Cu homeostatic cell type in the brain, and because liver is a major systemic Cu homeostasis organ, glia-specific and liver-specific KOs and KDs of Mblac1 are encompassed by the invention. For example, in a glial-specific Mblac1 KO or KD animal, Mblac1 gene expression is knocked out (eliminated) or knocked down (reduced), respectively, selectively in glial cells. By using a glial-specific Mblac1 KO or KD animal, agents can be evaluated for rescue of phenotypes derived from Cu+ dyshomeostasis more selectively than when deficits arise from a full body change in Mblac1.

Methods of knocking out and knocking down gene expression, including knocking out and knocking down expression selectively in a specific tissue, are well known in the art and customized KO rodents are commercially available from, e.g., The Jackson Laboratory (Bar Harbor, Maine). A description of how Mblac1 KO animals were made for use in the experiments described below is found in U.S. patent application Ser. No. 16/056,988, incorporated by reference herein. Gene knockout methods are also described in, e.g., U.S. Patent Applications pub. Nos. 20180020646, 20180105835, 20100107263, and 20100050277, all incorporated by reference herein. In some of the experiments described below, a swip-10 C. elegans model was used. In vivo screening methods using KO strains of C. elegans are known in the art, and described in, e.g., U.S. Pat. No. 7,531,713, incorporated by reference herein. Generally, when using KD or KO animals, one manipulates protein levels or activity of MBLAC1 either genetically (e.g., a mutation, siRNA) or chemically (e.g., with a drug), and one then measures what happens downstream regarding Cu and Cu-dependent processes and targets (e.g., oxidative stress, Cu-dependent enzyme activity, Cu-dependent mitochondrial functions, neural death, behaviors sensitive to brain Cu such as locomotor activity). Generally, in such methods, one is trying to normalize the KD or KO animals (with regard to Cu homeostasis).

### Methods to Identify Agents or Manipulations that Modulate Cu Dyshomeostasis in an Mblac1- or swip-10-Dependent Manner in a Subject

Described herein are methods to identify agents or manipulations that modulate Cu dyshomeostasis in an Mblac1- or swip-10-dependent manner in a subject. These methods are useful, for example, for identifying therapeutic agents that can restore Cu homeostasis in subjects suffering from Cu dyshomeostasis and in subjects at risk for developing Cu dyshomeostasis or associated disorders. Generally in these methods involving a genetic model of swip-10/Mblac1 WT and swip-10/Mblac1 KO/KD animals, a group comparison of WT to KO/KD animals (or cells/tissue therefrom) is performed. There are differences between the WT and KO/KD animals in terms of Cu+ levels, Cu-sensitive or Cu-responsive nucleic acids, proteins, peptides, and Cu-sensitive properties such as, e.g., mitochondrial function, oxidative stress, gene expression networks that are sensitive to Cu, etc. These differences are used to identify a medication that will eliminate or reduce the differences between the WT and KD/KO animals, i.e., bring the KO/KD levels to the WT levels (e.g., convert a KO/KD phenotype to a WT phenotype).

In an embodiment of a method to identify agents or manipulations that modulate Cu dyshomeostasis in an Mblac1- or swip-10-dependent manner in a subject, the method includes providing a plurality of Mblac1 or swip-10 KD or KO animals, and a plurality of Mblac1 or swip-10 WT animals. A first portion of the plurality of Mblac1 or swip-10 KD or KO animals and a first portion of the plurality of the Mblac1 or swip-10 WT animals are exposed to at least one test agent or manipulation, and a second portion of the plurality of of the Mblac1 or swip-10 WT animals are exposed to a control vehicle or manipulation. This results in four groups of animals: WT vehicle-treated, WT test agent-treated, KO/KD vehicle treated, and KO/KD test agent treated. Samples are collected from each animal, and a value for at least one Cu marker is measured in the samples from all the animals and the effects of the at least one test agent or manipulation between the first portion of the plurality of Mblac1 or swip-10 KD or KO animals and the second portion of the plurality of the Mblac1 or swip-10 WT animals are compared. The at least one test agent or manipulation is identified as one that modulates Cu dyshomeostasis in a subject if any of the following are observed: the value for the at least one Cu marker from the first portion of the plurality of Mblac1 or swip-10 KD or KO animals is statistically different from the value for the at least one Cu marker from the second portion of the plurality of Mblac1 or swip-10 KD or KO animals; the value for the at least one Cu marker from the first portion of the plurality of Mblac1 or swip-10 WT animals is statistically different from the value for the at least one Cu marker from the second portion of the plurality of Mblac1 or swip-10 WT animals; and the value for the at least one Cu marker from the first portion of the plurality of Mblac1 or swip-10 KD or KO animals is equal or similar to the value for the at least one Cu marker from the first and/or second portions of the plurality of the Mblac1 or swip-10 WT animals.

Modulating Cu dyshomeostasis includes alleviating Cu dyshomeostasis such that Cu homeostasis is normalized in the subject (an altered Cu response in the KO/KD animals is normalized). Modulating Cu dyshomeostasis also includes increasing (worsening) Cu dyshomeostasis. The step of measuring a value for at least one Cu marker in the samples from all the animals and comparing the effects of the at least one test agent or manipulation typically includes measuring a change in one of the following relative to pre-exposure: one or more Cu redox states, a Cu-dependent enzyme, a Cu-binding protein, a Cu-dependent physiological response or behavior, a Cu-dependent process, and a change in RNA, protein, posttranslational protein modification, or metabolite linked to Cu homeostasis.

In the methods, the samples can be any bodily fluid, tissue, organ or cell. Examples include peripheral tissue, a bodily fluid such as serum, feces, etc. Typically, a sample is one that can be used to monitor systemic Cu homeostasis (e.g., in the gut, liver and kidney). The Mblac1 or swip-10 WT animals and KO/KD animals can be any suitable animal model, e.g., rodents such as mice and rats, and C. elegans worms. The at least one Cu marker is typically one or more redox states of copper, a Cu-dependent enzyme, a Cu regulating process (e.g. H3 histone Cu reductase activity), Cu-binding protein, a Cu-dependent physiological response or behavior, a Cu-dependent process, or a change in RNA, protein, posttranslational protein modification, or metabolite linked to Cu homeostasis.

In the methods described herein, any suitable test agent can be subjected to the methods. The at least one test agent is typically one of: a small molecule, drug, nucleic acid, protein, peptide, nanoparticle, virus, or viral vector. For example, a test agent could be a virus expressing MBLAC1 protein or other modifying peptide. Another example of a test agent is a Cu chelator or a Cu chaperone that may rescue or mimic changes in Cu+ levels and phenotypes associated with Cu+ dyshomeostasis. Additional examples of test agents include enzymes that bind Cu, proteins that remove Cu, Cu transporters, agents that modify the enzymatic activity of Cu in a swip-10/Mblac1-dependent manner, histone Cu reductase modifying agents, and histone expression modifying agents that act to modify processes in a swip-10/MBLAC1-dependent manner. The at least one test agent can be a plurality of test agents in a library, e.g., a compound library. The at least one test agent can be detectably labeled. The at least one manipulation is typically one or more of: a genetic manipulation (manipulation of the subject's genome), a transcriptome manipulation (manipulation of the subject's transcriptome), a metabolome manipulation (manipulation of the subject's metabolome), and an environmental manipulation (e.g. mitochondrial or behavioral stress) linked to Cu dyshomeostasis.

In an alternative embodiment of the method, one measures a process, molecule and/or behavior known to be Cu-sensitive in its expression, localization or activity. For example, one could measure the animal's Cu-dependent behavior or Cu-dependent physiology monitored in vivo or measure Cu via an inserted microdialysis membrane and then euthanize the animals to end the experiment and save the brain/blood or other tissue from the animals for later analyses. The different groups of animals could also be given a drug and then either Cu-dependent physiology or behavior is measured and then the experiment is terminated (animals are euthanized) to either obtain postmortem data that cannot be obtained with living animals or simply to end the experiment.

In another embodiment of a method to identify agents or manipulations that modulate Cu dyshomeostasis in an Mblac1- or swip-10-dependent manner in a subject, the methods involve a genetic model of swip-10/Mblac1 WT and swip-10/Mblac1 KO/KD animals, and a series of groups of WT and KO/KD animals are being examined over time. Generally, baseline measurements are taken of all animals swip-10/Mblac1 WT and swip-10/Mblac1 KO/KD), then the animals are subjected to a manipulation such as a neural toxin or other neural insult that causes pathology (disruption of Cu homeostasis), then a potential treatment (e.g., a test agent or test drug) is given to a subset of animals followed by additional measurements, and a comparison is made between the treated subset and the untreated animals, and between the treated animals'measurements and the baseline measurements of those same animals. Typically, each of the 2 genotypes (i.e., swip-10/Mblac1 WT and swip-10/Mblac1 KO/KD) will have a distinct response to the manipulation, and when testing a potential treatment, the potential treatment is shown to be therapeutic if Cu homeostasis or its consequences is restored in the KO/KD animals such that it is equal or close to the Cu homoeostasis or its consequences seen in the WT animals.

In an example of such an embodiment, the method includes providing a plurality of Mblac1 or swip-10 KD or KO animals, and a plurality of Mblac1 or swip-10 WT animals. A baseline value is measured for at least one Cu marker for each animal. After measurement of the baseline value, each animal is exposed to at least one test agent or manipulation. In this method, each animal provides its own baseline value prior to exposure. At a first post-exposure time point, at least one exposure response value is measured for the at least one Cu marker or its consequences for each animal. In some embodiments, at one or more of a second, third, fourth, fifth and sixth post-exposure time point, an exposure response value is measured for the at least one Cu marker or consequence for each animal. In such embodiments, a time course of the exposure response to the at least one test agent or manipulation is determined. In some embodiments, the at least one test agent is detectably labeled. By detectably labeling the at least one test agent, identification of agents or manipulations that modulate Cu dyshomeostasis in an Mblac1- or swip-10-dependent manner in a subject can be facilitated. In some embodiments of the methods described herein, the at least one test agent is from a compound library, each member of the library identified by its position in storage plates prior to application. In other embodiments, the at least one test agent is tagged (e.g. bar coded with a DNA sequence) such that post-analysis evaluation would designate which drug or other reagent provided effective normalization (restoration of Cu homeostasis). Typically the steps of measuring a baseline value for at least one Cu marker or consequence and measuring at least one exposure response value for the at least one Cu marker include collecting a sample from the animals, in vivo imaging of a tissue (e.g., brain) in the animals, or measuring physiological behavior of all animals. For example, these measuring steps can include analyzing blood from the animals (and measuring the at least one Cu marker in the blood) and analyzing Cu-dependent behavior in the animals In this example, analyzing both a blood sample for a Cu marker and the animal's behavior can provide validation and increased specificity in a scenario in which the animal displays a Cu-dependent behavior but many things show this behavior that are not Cu dependent. So the combination of a positive behavioral finding with a blood measure that also can indicate altered Cu homeostasis can provide a better conclusion than either alone.

As referred to above, glia-specific and liver-specific KOs of Mblac1 can be used in the methods described herein. Such models are useful in the methods because rescue of deficits by expression of wildtype swip-10 in glial cells was shown, because glia are a major homeostatic cell type in the brain for Cu, and because liver is a major systemic Cu homeostasis organ.

Generally in these methods, values for the at least one Cu marker are measured from baseline samples and from post-exposure or post-treatment samples from all animals, and one compares the effects of the at least one test agent or manipulation between each group of animals. For example, in some embodiments of this method, several comparisons are made: 1) WT vs. KO/KD at baseline before exposure or treatment, 2) KO/KD at any point after exposure or treatment compared to its own pre-exposure or pre-treatment, 3) WT at any point after exposure or treatment compared to its own pre-exposure or pre-treatment, and 4) KO/KD at any point can be compared to WT at any point. From such comparisons, any deviation of or difference between WT and KO/KD indicates that the tested agent or manipulation modulates SWIP-10/MBLAC-1 dependent Cu dyshomeostasis. Typically, a finding that the values in the post-exposed or post-treated KO/KD animals are closer statistically to the values in the pre-exposure or pre-treatment WT animals than to the values in the pre-exposed or pre-treated KO/KD animals indicates that the tested agent or manipulation restores Cu homeostasis.

Methods to identify agents or manipulations that modulate Cu dyshomeostasis in an MBLAC1- or SWIP-10-dependent manner include encompass methods of identifying agents or manipulations that modulate Cu dyshomeostasis in an MBLAC1- or SWIP-10-dependent manner in cells obtained or derived from a subject (e.g., a human subject, Mblac1 or swip-10 KD or KO animals). Cells obtained or derived from KD and KO animals as described herein can be used as a platform for screening for agents that modulate (e.g., restore) Cu dyshomeostasis in an Mblac1- or swip-10-dependent manner. Typically, the cells are obtained or derived from the KD and KO animals, they are cultured, and then subjected to one or more test agents to determine whether Cu-dependent or Cu-related measures they exhibit can be normalized by the one or more test agents. In an embodiment, cells derived from an Mblac1 KO mouse, such as glia or hepatocytes, are compared to the same cells taken from Mblac1 WT animals. These cell comparisons are analogous to the animal comparisons as they can exhibit differences in many of the same measures (except animal behavior). Because it was shown that embryonic fibroblasts show oxidative stress as compared to fibroblasts taken from WT animals, these cells can be screened for agents that normalize the Mblac1 mutants (Mblac1 KO or KD). In some embodiments, the methods described herein are performed using both KO or KD animals (in vivo experimentation) and cells from those animals (in vitro experimentation).

A non-limiting list of examples of cell types that can be used in the methods includes: primary cell cultures used for short term studies, cells transformed with a virus to immortalize them for long term use (i.e., immortalized cells), stem cells (that can differentiate into cells forming different organs can be used so as to model changes in these tissues), cells grown as monolayers on culture plates or in suspension for non-adherent cells (e.g, immune cells), mixed cell cultures, cell cultures made up of specific combinations of different cells, cells grown as cell aggregates with the ability to generate 3D structures (e.g., as organoids), mixed cell cultures of neurons and glia, glial cells, neuronal cells, hepatocytes, cardiac cells, differentiatable embryonic stem(ES) cells, and muscle cells. In embodiments, organoids that contain both glia and neurons and thus can be more reflective of the in vivo circumstance are used in a screen to identify agents or manipulations that modulate Cu dyshomeostasis in an MBLAC1- or SWIP-10-dependent manner.

### Method to Identify Agents that Support Cu-Dependent Neuronal Health in a Subject

Also described herein are methods to identify agents that support Cu-dependent neuronal health in a subject. Cu-dependent neuronal health generally is assessed by evaluation of neuronal structure, neural signaling, neural excitability, or neural metabolism and oxidative stress. The methods include use of Mblac1 or swip-10 KD or KO animals and cells obtained or derived from the animals. A typical method includes providing a plurality of Mblac1 or swip-10 KD or KO animals, and a plurality of Mblac1 or swip-10 WT animals. A first portion of the plurality of Mblac1 or swip-10 KD or KO animals and a first portion of the plurality of Mblac1 or swip-10 WT animals are exposed to a drug, neural toxin or other neural insult that results in pathology in a Cu-dependent manner. A second portion of the Mblac1 or swip-10 KD or KO animals and a second portion of the Mblac1 or swip-10 WT animals are not exposed to the neural toxin or neural insult and serve as controls. The pathology in each exposed animal is measured followed by administration of a test agent to at least the first portion of the plurality of Mblac1 or swip-10 KD or KO animals and the first portion of the plurality of Mblac1 or swip-10 WT animals. The pathology in each animal that was administered the test agent is measured. The test agent is identified as an agent that supports Cu-dependent neuronal health in a subject if the test agent reverses the pathology of an Mblac1 KO, mimicking the effects of other genes linked to Cu+ dyshomeostasis or drugs that induce Cu+ dyshomeostasis (e.g. Cu+ chelator) or rescued by Cu+ supplementation as with administration of a Cu+ chaperone.

In these methods, measuring the pathology in the animals can include one or more of visual, biochemical, physiological and behavioral measurement of a marker of neuronal damage in the animals. In some embodiments, the identified agent supports Cu-dependent neuronal health in the presence of MBLAC1 but not in the absence of MBLAC1. Typically, the neural toxin or other neural insult induces oxidative stress and/or neural degeneration in the animals. In embodiments, the identified agent has neuroprotective activity when administered to a mammalian (e.g., human) subject in need thereof. In some embodiments, the plurality of Mblac1 or swip-10 KD or KO animals are swip-10 KD or KO C. elegans animals, and the plurality of Mblac1 or swip-10 WT animals are swip-10 WTanimals. Pathology can be measured by any suitable metric, e.g., by measuring proteins that are stimulated during cell death progression, by measuring the number of neural cells, or by an anatomical assay that counts healthy parts of a cell including dendrites or cell bodies.

In such methods, typically one is measuring visually, biochemically, physiologically or behaviorally something that is indicative of (a marker of) neuronal damage based on the experimental results described herein. From the experimental results it has been shown that in swip-10 worms, the neurons are shrunken and fragmented, they display changes in Cu+, elevated measures of oxidative stress and reduced measures of mitochondrial function, and they paralyze in water whereas WT worms do not, indicating functional alterations in neurons as they die. So any of these measures can be used in a method to identify agents that support Cu-dependent neuronal health as described herein, e.g., animals are chronically or acutely treated with a candidate drug to screen for agents that prevent or reverse these measures which are signs of poor neuronal health.

In another embodiment of a method to identify agents that support Cu-dependent neuronal health as described herein, WT or KO/KD animals are administered a drug or other manipulation (e.g. a toxic gene by crossing with another line of animals carrying the toxic gene) that produces signs of Cu-dependent neuropathology. If the KO/KD genotype makes the pathology-inducing drug's or other manipulation's effects worse, one can then screen for drugs that, when applied to the pathological agent-treated KO/KD and WT animals, eliminate the additive effect of the KO/KD genotype. This can be done as a function of dose of the pathological agent, where the KO/KD effect is to make pathology evident, and thus where the test (candidate) agent now makes neuronal health totally normal, like what it is when treated with subtoxic pathogen. Alternatively, this can be done as a function of age, treating WT animals with a toxin/pathogen that requires chronic treatment to induce pathology. If the animals are examined at a younger age, one would expect to see no pathology. But in the KO/KD, the toxic effect is made evident by the loss of SWIP-10/MBLAC1. Then one would screen the library of molecules (test agents) for any that revert the pathology of the KO/KD when treated with the toxin, at the younger age, as if there were not a KO/KD manipulation.

### Methods of Treating Diseases Associated with Cu Dyshomeostasis

Described herein are methods of identifying therapeutic agents that are neuroprotective, e.g., agents that alleviate Cu dyshomeostasis and support Cu-dependent neural health. Also described herein are methods of administering these agents to a subject in need of such treatment who has a disease or disorder associated with Cu dyshomeostasis. A disease or disorder associated with Cu dyshomeostasis can be a disease or disorder that has been reported to exhibit, any of, as examples, oxidative stress, mitochondrial dysfunction, neural degeneration, etc. Examples of diseases associated with Cu dyshomeostasis include AD, PD, Menkes disease, Wilson disease, fatal infantile cardioencephalomyopathy, metabolic syndrome, anemia, cardiovascular disease, cancer, neurodegenerative disease, diabetes, etc.

In such methods, a therapeutically effective amount of a therapeutic agent is administered to a subject (e.g., a human) having a disease or disorder caused by Cu dyshomeostasis. Typically, the therapeutically effective amount results in at least one of the following desirable results: induction or enhancement of mitochondrial respiration, enhancement (promotion) of neural cell health, reduction of neural cell death, suppression of oxidative stress, and prolonging of survival in the subject. Such a therapeutically effective amount can be determined according to standard methods. Toxicity and therapeutic efficacy of an agent that supports Cu-dependent neural health as described herein, or compositions containing the agent, that are identified and/or utilized in the methods described herein can be determined by standard pharmaceutical procedures. As is well known in the medical and veterinary arts, dosage for any one individual depends on many factors, including the individual's size, body surface area, age, the particular composition to be administered, time and route of administration, general health, and other drugs being administered concurrently. A delivery dose of a therapeutic agent or composition as described herein is determined based on preclinical efficacy and safety. Administration of the therapeutic agent or a composition containing the therapeutic agent to the subject can reduce or eliminate Cu dyshomeostasis in the subject. In an embodiment, the subject suffers from elevated oxidative stress and administration of the therapeutic agent or a composition containing the therapeutic agent to the subject reduces oxidative stress in the subject. In another embodiment, the subject suffers from neural degeneration and administration of the therapeutic agent or a composition containing the therapeutic agent to the subject reduces neural degeneration in the subject. Such treatment will be suitably administered to individuals, particularly humans, suffering from, having, susceptible to, or at risk for a disease, disorder, or symptom thereof (e.g., a disorder characterized by Cu dyshomeostasis). Determination of those subjects or individuals “at risk” can be made by any objective or subjective determination by a diagnostic test or opinion of a subject or health care provider.

Any suitable methods of administering a therapeutic agent (e.g., neuroprotective agent) as described herein, or compositions containing the therapeutic agent, to a subject may be used. In these methods, the therapeutic agents and compositions containing therapeutic agents may be administered to a subject by any suitable route, e.g., oral, buccal (e.g., sub-lingual), intratumoral, parenteral (e.g., subcutaneous, intramuscular, intradermal, or intravenous), topical (i.e., both skin and mucosal surfaces, including airway surfaces), rectal, vaginal, and transdermal administration. In another embodiment, the therapeutic agents and compositions may be administered directly to a target site (e.g., the brain), by, for example, surgical delivery to an internal or external target site, or by catheter to a site accessible by a blood vessel. If administered via intravenous injection, the therapeutic agents and compositions may be administered in a single bolus, multiple injections, or by continuous infusion (e.g., intravenously, by peritoneal dialysis, pump infusion). For parenteral administration, the therapeutic agent or composition is preferably formulated in a sterilized pyrogen-free form.

### Methods of Identifying Molecular Networks

Also described herein are methods of identifying molecular networks in an animal that are altered by manipulation of Cu up or down (e.g., with a Cu chelator or chaperone) and that can be reduced or elevated by changing swip-10/MBLAC1 expression or MBLAC1 activity by pharmacological or genetic manipulations. The effort here is to evaluate which Cu sensitive molecular pathways rely on swip-10/MBLAC1 activity versus those influenced by other Cu reductases. In the methods animals are treated with the chelator or other Cu reducing agent, the animal is processed to measure transcriptome, proteome, metabolome networks, and in parallel, MBLAC1/swip-10 levels are elevated or reduced to identify the Cu dependent networks that are sensitive to MBLAC1/swip10 manipulation. These new components of the Cu modulated networks are defined as MBLAC1/swip-10 reducible/elevatable elements in that network and now can be targeted themselves for drug development or examined for contributions to pathology. Cu manipulation changes a lot of things. Manipulating MBLAC1/swip-10 will counter a portion of these effects. This portion is what one wants to identify as this portion is now a new target or is comprised of new targets that reflect the SWIP-10/MBLAC1 dependent Cu homeostatic pathway, a pathway now that is a new drug target. Methods of identifying molecular networks in an animal are described in U.S. patent application Ser. No. 16/057,013, incorporated by reference herein in its entirety.

### Additional Embodiments

Further provided herein are the agents identified as alleviating Cu dyshomeostasis and/or supporting Cu-dependent neural health and compositions containing such agents for use in the treatment of Cu dyshomeostasis, or a disorder associated with Cu dyshomeostasis (e.g., AD, PD), in a subject in need of such treatment. Examples of identified agents (e.g., therapeutic agents) include small molecules, proteins, peptides, nucleic acids, viruses, viral vectors and nanoparticles. Since MBLAC1 regulates histone production, agents used to regulate histones such as HDAC or HAT modulatory molecules could also be tested (screened) as possible agents for alleviating Cu dyshomeostasis and/or supporting Cu-dependent neural health. Because Cu chaperones can rescue the KO phenotypes described herein, Cu chaperones could also be tested (screened) as possible agents for alleviating Cu dyshomeostasis and/or supporting Cu-dependent neural health.

Also described herein is a method for screening for agents that bind to or modulate expression or function of MBLAC1 and can treat a disorder caused by Cu dyshomeostasis. The method typically includes providing purified MBLAC1 protein, MBLAC1-expressing cells, or extract from MBLAC1-expressing cells. In one example of the method, one identifies a molecule that binds to MBLAC1 or regulates its endonuclease activity, and then tests that reagent for its ability to modulate Cu dyshomeostasis using markers that were described above (e.g. Cu itself, Cu-dependent targets etc). In some methods, the agent is administered to an animal or cell or extract to determine if it normalizes Cu dyshomeostasis triggered by a mutation or pharmacological manipulation.

In the method, the disorder can be one that can be produced or rescued by Cu manipulations. In some embodiments of the method, Cu levels are reduced with a metal chelator molecule or a drug manipulation that alters Cu transport or eliminates Cu so that with induction of more MBLAC1 or higher activity MBLAC1, this Cu manipulation is without effect.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

By the terms “copper” and “Cu” are meant any form of the chemical element with the symbol Cu, including oxidation states of Cu (−2, 0,[2]+1, +2, +3, +4). In some embodiments, a Cu-containing compound, such as a salt, may be administered to a subject in need thereof.

As used herein, the terms “agents or manipulations that modulate Cu dyshomeostasis” mean any pharmacological agent, biological agent, chemical agent, or physical manipulation that produces a change in any Cu-related process. A “test agent or manipulation” is an agent or manipulation that is being tested for an ability to modulate (e.g., alleviate) Cu dyshomeostasis (e.g., by restoring Cu homeostasis).

By the phrase “in an MBLAC1- or swip-10-dependent manner” is meant any change that is produced by modifying MBLAC1/swip-10 expression any comparing to WT expressers or any change produced by a treatment in a WT condition but not in an MBLAC1/swip-10 modified condition, or any change produced by a treatment in an MBLAC1/swip-10 modified condition but not in WT.

As used herein, the term “Cu marker” means any state, condition, chemical or biological agent (expression, level or function thereof), or behavior that is affected by Cu in any of its forms, that affects Cu in any of its forms, and/or that interacts with Cu in any of its forms. Examples of a Cu marker include one or more Cu redux states, a Cu-dependent enzyme, a Cu-binding protein, a physiological behavior, a change in RNA (e.g., a level of a particular RNA sequence that is elevated or decreased), and a-dependent process. A Cu-dependent process is any outcome that is affected by the presence of Cu, including but not limited to Cu-dependent enzymes that mediate synthesis of products, alter levels of molecules of oxidative stress.

By the phrases “normalizing Cu homeostasis” and “restoring Cu homeostasis” is meant manipulating an organism having Cu dyshomeostasis, for example, by administering an agent or intervention to the organism, that results in the organism achieving Cu homeostasis. For example, the methods described herein include screening for (identifying) agents and manipulations (interventions) that can transform a state of Cu dyshomeostasis in an organism to a state of Cu homeostasis in the organism.

As used herein, the phrase “Cu-dependent neuronal health” means any outcome that is altered by a change in Cu levels and influences neruonal health indices that include but are not limited to oxidative stress, neurodegeneration, normal levels of enzymes, neurotransmitters.

An agent that has “neuroprotective activity” is any agent that decreases/reverses any process that is adverse to neuronal health.

By the term “peripheral tissue” is meant any organ or body fluid that does not include the brain and spinal cord or its surrounding fluids (e.g. cerebral spinal fluid). Examples of peripheral tissue include liver and kidney.

The term “modulate” means to regulate or adjust, e.g., to decrease or increase a measure, parameter, level, concentration, etc.

As used herein, “sample” is typically a biological sample obtained from an organism. A biological sample can be, e.g., cells, blood, serum, sputum, tissue, saliva, cerebrospinal fluid, cellular or tissue extract, etc. Cells or tissue from any organ, including liver and kidney, can be used as a biological sample.

The terms “agent” and “therapeutic agent” as used herein refer to a chemical entity or biological product, or combination of chemical entities or biological products, administered to a subject to treat a disease or condition (e.g., a disorder associated with Cu dyshomeostasis such as AD or PD). Examples of agents include drugs such as small molecule drugs and biologics (e.g., nucleic acids, proteins, peptides, antibodies, nanoparticles, viruses). In addition to reversing dyshomeostasis, agents can also be used to explore the mechanism, but not reverse. For example, when one gives an agent that one thinks will affect WT but not affect MBLAC1 KO because its actions require a pathway downstream of MBLAC1 protein activity and thus may only show an influence with WT animals.

The terms “patient,” “subject” and “individual” are used interchangeably herein, and mean a subject to be treated, diagnosed, and/or to obtain a biological sample from. Subjects include, but are not limited to, humans, non-human primates, horses, cows, sheep, pigs, rats, mice, dogs, cats, worms, fish, and other animals. A human in need of Cu dyshomeostasis treatment is an example of a subject. A human who is at risk for Cu dyshomeostasis is another example of a subject.

As used herein, the terms “treatment” and “therapy” are defined as the application or administration of a therapeutic agent or therapeutic agents to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

All publications, patent applications, and patents mentioned herein are incorporated by reference in their entireties. In the case of conflict, the present specification, including definitions, will control. The particular embodiments discussed below are illustrative only and not intended to be limiting.

### EXAMPLES

The present invention is further illustrated by the following examples. The examples are provided for illustration only and should not be construed as limiting the scope of the invention in any way.

### Example 1—Targeting SWIP-10 for the Therapeutic Enhancement of Cu Dependent Mitochondrial Respiration, Reduction Oxidative Stress and Reduction in Neurodegeneration

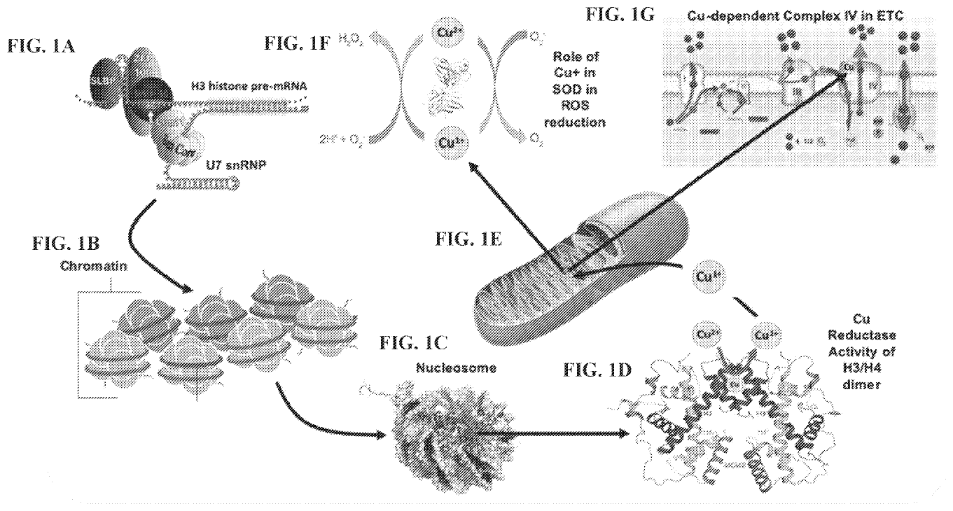

The hypothesis that: 1) SWIP-10, through its support of histone H3 mRNA processing, may be required to ensure adequate levels of H3 protein and thereby to sustain production of Cu+; and 2) reduced Cu+ availability might then reduce mitochondrial function, elevate oxidative stress and elevate risk for neurodegeneration, was investigated. A model for SWIP-10 in Cu+ dependent regulation of mitochondrial energetics and suppression of oxidative stress is shown in FIG. 1A-1G.

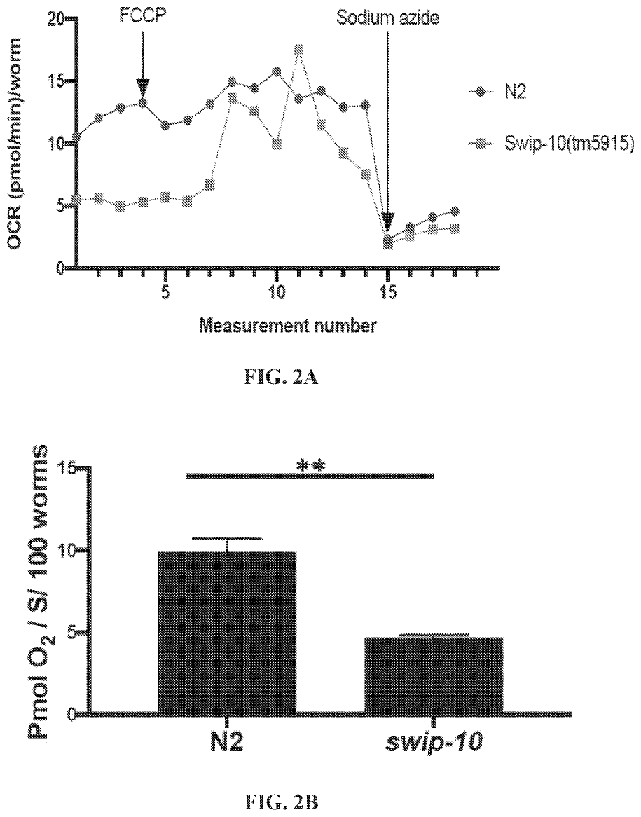

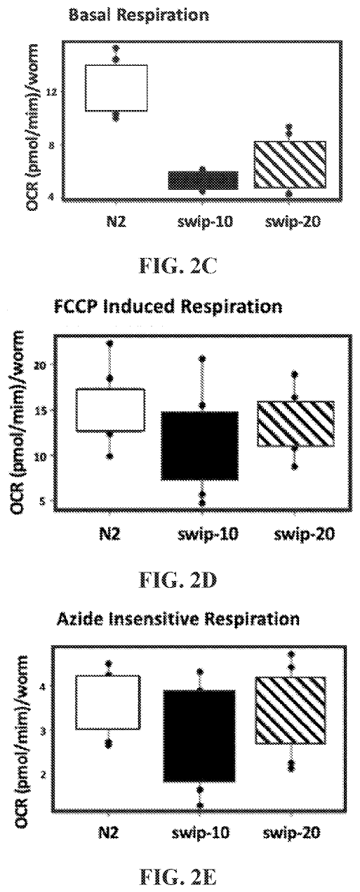

Evidence that SWIP-10 plays a role in mitochondrial respiration or Cu+ homeostasis in vivo in a model undergoing neurodegeneration is shown in FIGS. 2A and 2B (which relate to mitochondrial respiration. This change in living nematodes using two different measures of oxygen consumption is shown in the data of FIGS. 2A and 2B, showing that a genetic loss of swip-10, which causes dopamine neuron degeneration, produces a significant deficit in basal mitochondrial respiration (FIG. 2A—measurements 0-7) that can be bypassed using FCCP, a drug that acts to stimulate the oxidative respiration pathway at a step (Complex V) beyond Cu+ dependent COX (Complex IV). Similar deficits in basal respiration were seen with two different instruments (FIGS. 2A and 2B). Because wildtype and swip-10 animals exhibit equal rates of maximal respiration (FIG. 2A measurements 8-12), these experiments also indicate that mitochondrial number and structure are likely to be unperturbed, consistent with loss of SWIP-10 producing a Cu+ dependent functional deficit in mitochondrial respiration.

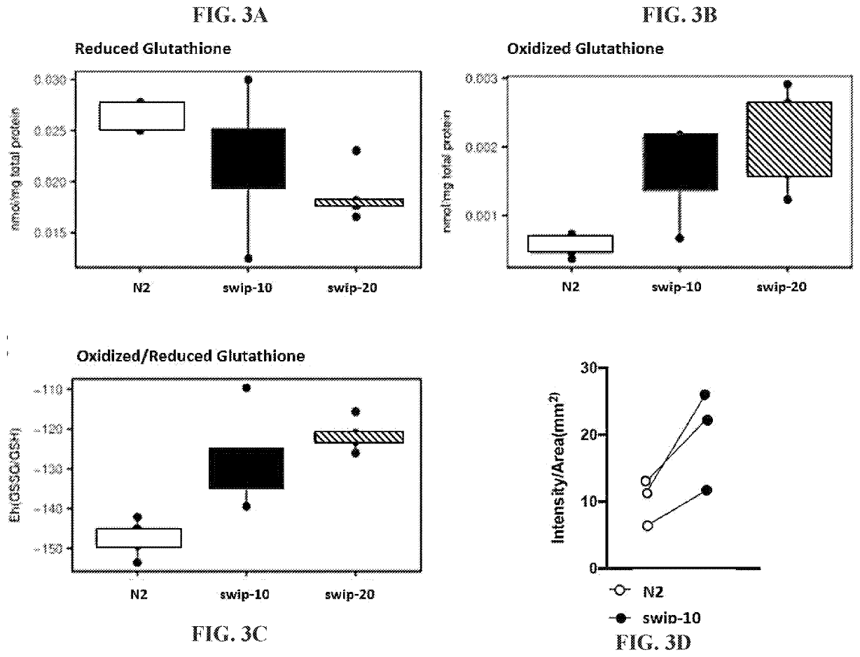

To obtain a more direct measure of oxidative stress, HPLC-based approaches were used to investigate the oxidative state of the major cellular buffer for oxidative stress, glutathione (FIG. 3A-3D). Glutathione in its reduced state (GSH) can detoxify oxidative free radicals and peroxides via conversion to the oxidized form GSSG where a disulfide is formed between two formerly reduced GSH molecules. Under states of oxidative stress, levels of reduced GSH are diminished whereas levels of oxidized GSSG are increased. The “redox potential” can be calculated from the ratio of oxidized to reduced glutathione and is expected to become elevated under oxidative stress conditions. As shown in FIG. 3A-3D, swip-10 mutant animals display elevated levels of GSSG and an increased redox potential, demonstrating biochemically that these animals are under a state of global oxidative stress.

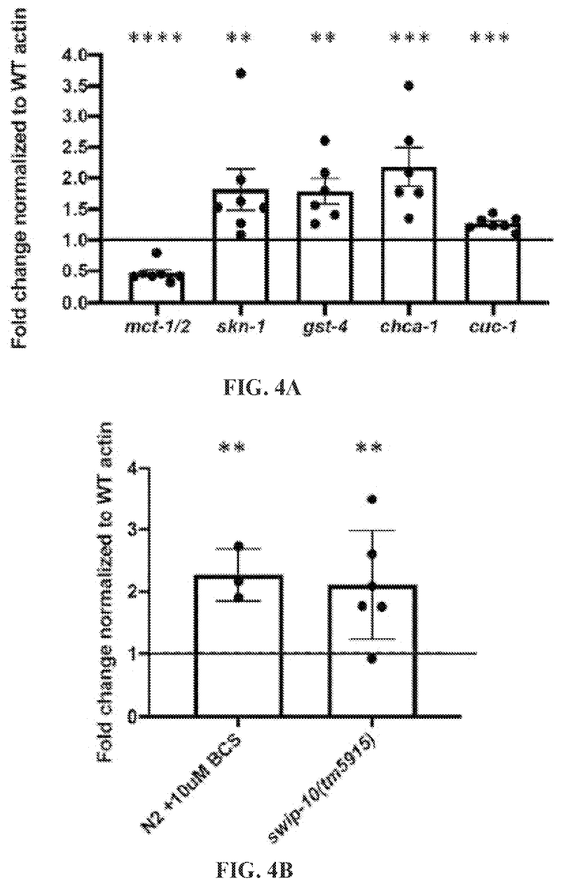

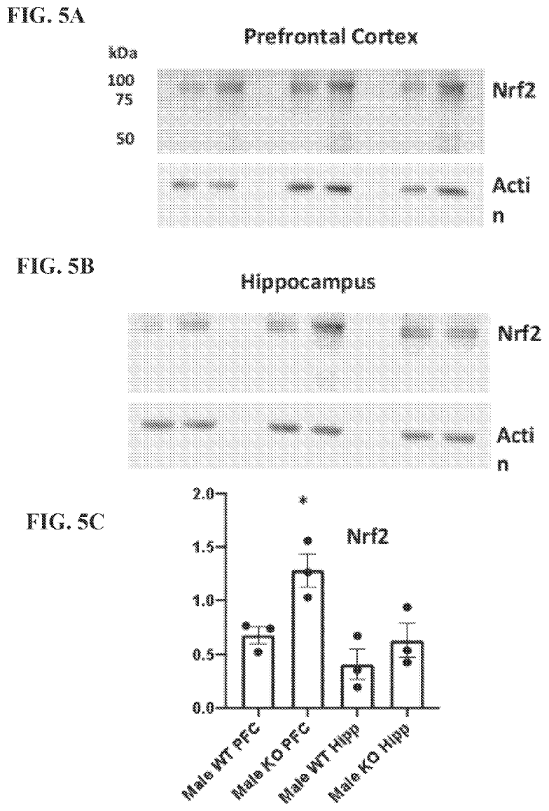

If swip-10 mutant animals are under Cu+-dependent mitochondrial and oxidative stress, they should display changes in the expression of genes linked to cellular energetics, oxidative stress, and Cu+ homeostasis. Using a qPCR approach, expression of genes that report such changes was quantified, with results shown in FIG. 4. Levels of the monocarboxylate transporter (mct-1/2) that transfers lactate and pyruvate between cells was found to be significantly diminished in swip-10 mutants. Lactate and pyruvate are often shipped between cells as precursors to the Krebs cycle in metabolically active cells for metabolism by the mitochondrial electron transport chain, synthesizing ATP. Reduced expression of mct-1/2 can be interpreted as a response to a reduction in metabolic function: a compensatory mechanism to diminish cellular loss of lactate and pyruvate, now needed within cells to offset diminished mitochondrial function. It was also found that gst-4 and skn-1 mRNAs were elevated in swip-10 animals. Expression of the gst-4 gene is upregulated by oxidative stress, as is skn-1, the worm ortholog of Nrf2. These findings are consistent with swip-10 mutants being under oxidative stress, as shown with the elevation in the redox potential. As shown in FIG. 5, like swip-10 worms, brain tissues from Mblac1 KO mice show elevations in Nrf2, in this case at the protein level, providing evidence that similar changes are instituted in both worm and mouse models, A glutamate transporter gene, glt-1, was also found to be elevated, which is of interest as in rodents, treatments with ceftriaxone, a drug that binds to MBLAC1, triggers an elevation of the GLT1 type glutamate transporter (Rothstein et al, Nature 2005). A highly significant elevation in the Cu+ transporter chca-1 was observed in swip-10 mutants. The chca-1 gene is the worm ortholog of the mammalian Cu+ transporter gene CTR1 and has been shown to be upregulated by Cu+ deficiency (Yuan et al, J. Biol Chem 2018). These findings provide support that loss of production of SWIP-10 protein, per the model (FIG. 1) of reduced histone H3 protein expression, leads to diminished Cu+, altering mitochondrial function and oxidative stress, and triggering a compensatory elevation of CHCA-1 expression to attempt to acquire more Cu+ from peripheral sources.

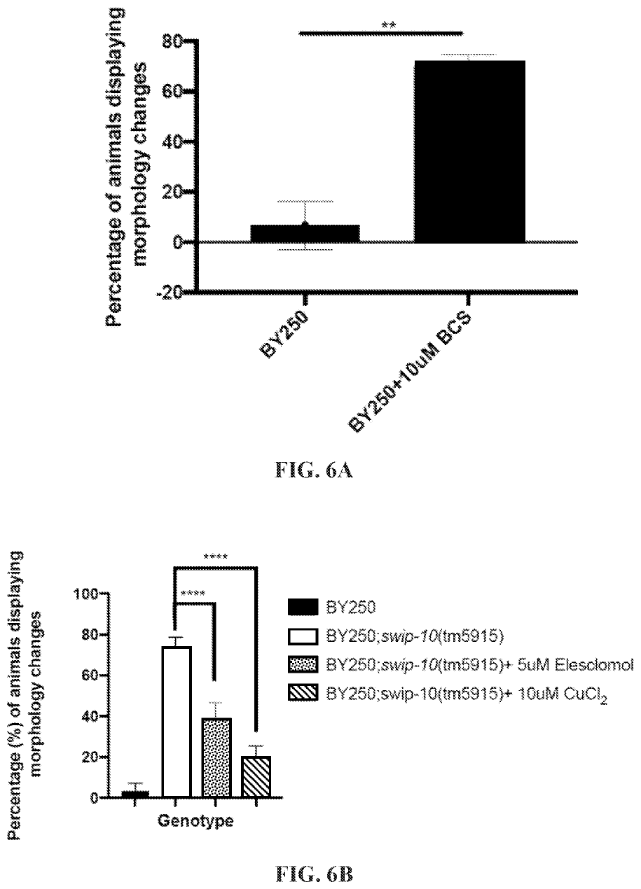

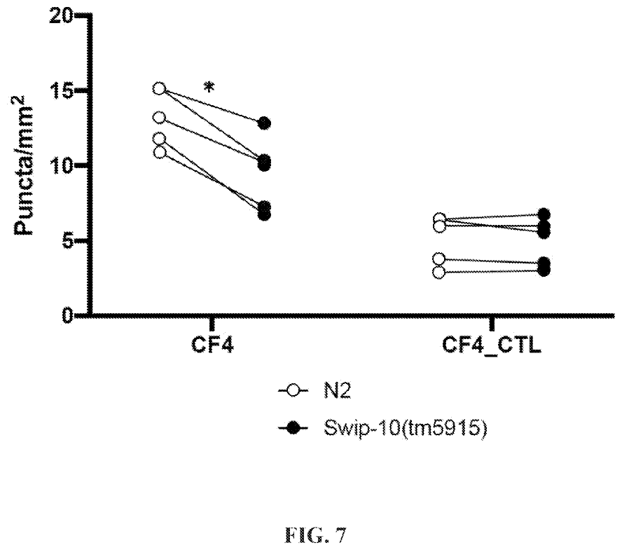

Sustained alterations in mitochondrial function and increased oxidative stress are expected to place neurons at risk for death due to their high metabolic rate. Neurodegeneration was quantified in WT and swip-10 mutants via confocal imaging of DA neurons after a cross of these lines to a line (BY250) that possesses GFP-labeled DA neurons. Assays were performed blind to genotype and treatment, and degeneration assessed by scoring the frequency of dendritic breaks, shrunken soma and missing neurons as described by Gibson et al, 2018. Extending work examining degeneration in BY250: swip-10 vs BY250, a potential Cu-sensitive regulation of swip-10 degeneration was assessed using growth of BY250 animals on the Cu+ specific chelator, BCS, to promote degeneration, and incubation of BY250: swip-10 on CuCl2 and elesclomol to prevent swip-10 degeneration. FIGS. 6A and 6B show results from these studies. FIG. 6A shows evidence of DA neuron degeneration generated by incubation of WT on the Cu+-specific chelator BCS, whereas in FIG. 6B, it can be seen that degeneration of swip-10 was rescued by incubation of swip-10 animals with either CuCl₂ or the Cu+ chaperone elesclomol. Importantly, as with rescue of dopamine neuron degeneration shown here, boosting Cu levels with elesclemol has been found to reduce pathology associated with Menkes disease, a condition of insufficient Cu+ availability.

### Example 2—Targeting SWIP-10/MBLAC1 for the Therapeutic Enhancement of Cu Dependent Mitochondrial Respiration, Reduction Oxidative Stress and Reduction in Neurodegeneration and In Peripheral Tissue

Above it was shown that swip-10 mutants demonstrate elevated expression of the oxidative stress-sensitive transcription factor skn-1, worm ortholog of mammalian Nrf2. The antibiotic ceftriaxone (Cef) binds MBLAC1 and demonstrates neuroprotection in animal models of AD and other neurodegenerative disorders, stroke, oxygen glucose deprivation induced neurodegeneration, and motor neuron degeneration. (Rothstein et al. Nature. 2005; 433:73-7). The neuroprotective effects of Cef have been demonstrated to require the induction of Nrf2 (Lewerenz et al., J Neurochem. 2009; 111:332-43).

To provide support that loss of MBLAC1 exerts effects that parallel those seen with loss of SWIP-10, two analyses were performed in MBLAC1 KO mice. The first was an assessment of whether MBLAC1 KO mice demonstrate elevations in Nrf2 expression, paralleling the findings with swip-10 mutants, where an elevation of expression of the Nrf2 ortholog skn-1 was found (FIG. 3A-3D). The antibiotic ceftriaxone (Cef) binds MBLAC1 and demonstrates neuroprotection in animal models of AD and other neurodegenerative disorders, stroke, oxygen glucose deprivation-induced neurodegeneration, and motor neuron degeneration. (Rothstein et al. Nature. 2005; 433:73-7). The neuroprotective effects of Cef have been demonstrated to require the induction of Nrf2 (Lewerenz et al., J Neurochem. 2009; 111:332-43). As shown in FIG. 5A, when extracts of cortical tissue from 22 wk old Mblac1 KO mice are immunoblotted for NRF2 protein and these levels were compared to levels found in WT littermate mice, a significant elevation in NRF2 protein expression was found in KOs. An elevation of NRF2 protein was also seen in hippocampus (hipp) (FIG. 5B), though this increase did not reach statistical significance (FIG. 5C).

In the second, particularly to determine whether mitochondrial respiration is affected in peripheral tissues of Mblac1 KO mice, the levels of mitochondrial coenzymes NADH and FAD, and their redox ratio (NADH/FAD, RR), were quantified in peripheral tissues (kidneys and livers) of WT and homozygous Mblac1 KO littermates, using 3D optical cryo-imaging (B. Ceyhan et al., “Optical imaging reveals liver metabolic perturbations in Mblac1 knockout mice” Engineering in Medicine and Biology Conference, 2023). In this methodology, the NADH and FAD intensities and the RR are direct reflections of mitochondrial activity. The optical cryo-imaging method was useful for visualizing and quantifying changes in metabolic state in WT and Mblac1 KO mice. It was found that Mblac1 KO mice exhibited a greater oxidized redox state compared to WT mice. When compared to the WT group, the RR of livers from Mblac1 KO mice was decreased by 46.32% (statistical analyses showed a significant difference between the WT and KO groups with p<0.05), driven predominantly by significantly lower NADH levels (a more oxidized state). Thus, mitochondrial respiration is altered in Mblac1 KO mice indicative of an oxidized environment. These findings are consistent with the presence of peripheral comorbidities accompanying neurodegenerative disease such as CVD that occurs in AD in the context of reduced MBLAC1 expression.

Next, whether Cu+ levels are diminished in cells from swip-10 mutant worms was investigated. Cu+ was assessed using confocal imaging (Nikon AIR, FAU Cell Imaging Core) with animals treated with the Cu+ specific fluorophore CF4 versus a Cu+ insensitive version of the sensor (Xiao et al, Nature Chem Bio, 2018). In these studies, 12-16 hours prior to imaging at the L4 stage, animals were collected from plates, washed in M9 solution, then treated with 25 μM of either CF4 or control CF4 probe by incubation in tubes rocking overnight. Intensity values from each were averaged across at least 10 animals and analyzed statistically via two-way ANOVA (WT, swip-10, +/−CF4 and Cu+ insensitive fluorophore). In FIG. 7, evidence is presented that swip-10 mutants demonstrate diminished Cu+ levels using CF4, with no genotype effect seen using the Cu+ insensitive CF4-like fluorophore. Unlike worms, it is not possible to use the CF4 approach to evaluate Cu+ levels in the whole body of mammals. Changes in peripheral organ metal levels, including Cu, were found from Mblac1 KO mice, specifically Cu and Zinc in kidney, though the tools used to date do not discriminate the different oxidation states of Cu.

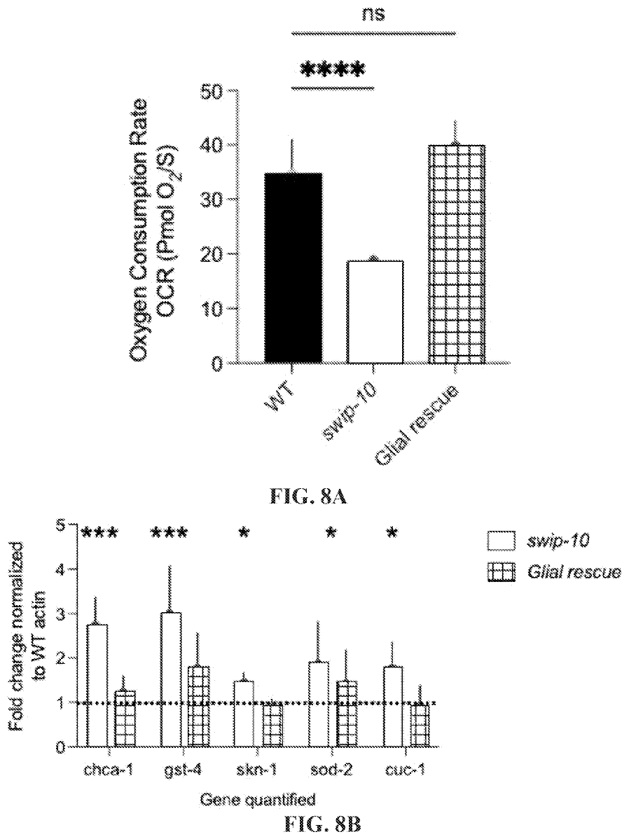

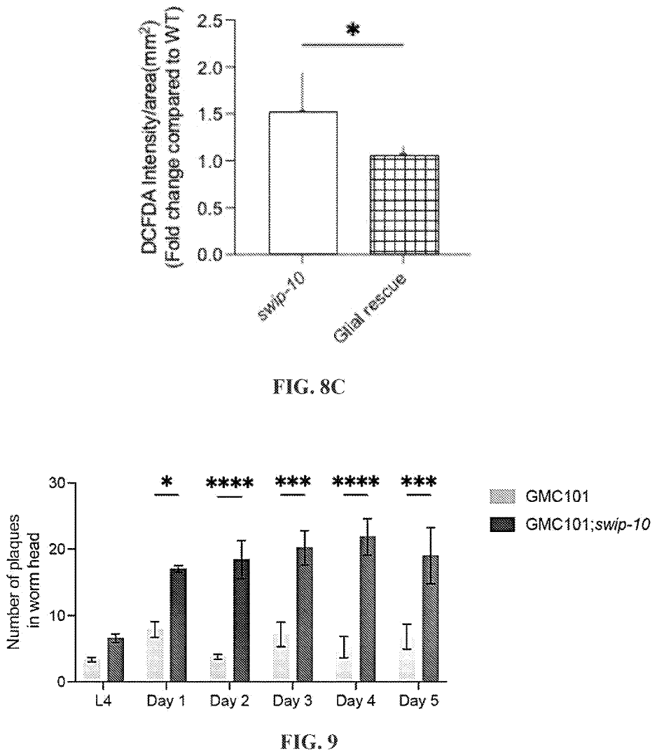

Since glial cells are known to physically and functionally interact with neurons, supporting neural signaling and viability, it seemed likely that the effects described above in swip-10 mutants were due to Cu+ dependent alterations that are initiated in glial cells. Experiments were thus performed to show that reduced mitochondrial function, elevated gene expression, and oxidative stress are suppressed by expression of wild type swip-10 selectively in glial cells, using the pan glial promoter ptr-10. The basal OCR, indicative of mitochondrial function, was determined by the method of Oroboros Oxygraph respirometer as an average of steady state recordings over 10 minutes. One-way ANOVA used for analysis. OC is decreased in swip-10 animals, and this is prevented with glial rescue using the ptr-10 promoter to drive swip-10 expression selectively in glia (FIG. 8A; **p≤0.01). Gene expression changes in ROS- and Cu-related genes quantified via qPCR were also normalized by glial swip-10 expression (FIG. 8B). Finally, increased ROS levels in swip-10 mutants, indictated with the fluorescent ROS sensor, DCFDA, were normalized with glial swip-10 expression (FIG. 8C).

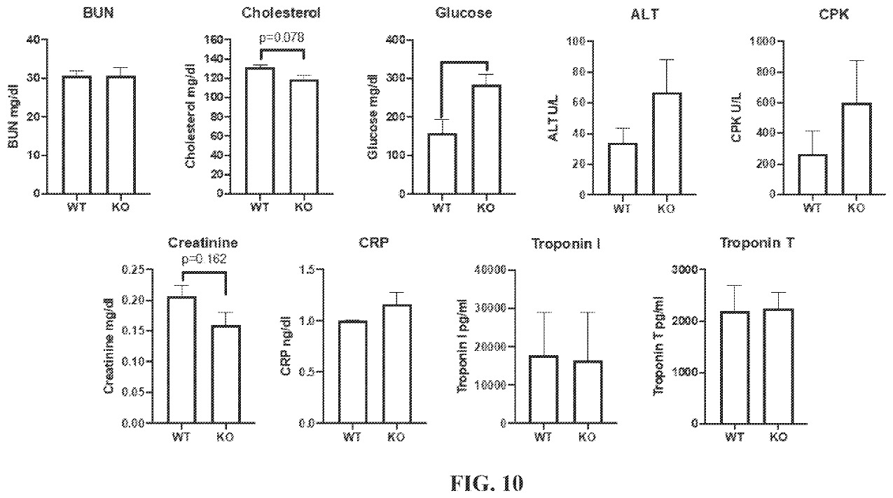

The neurodegeneration observed in swip-10 mutants raised the possibility that swip-10 loss might demonstrate a change in a physical measure seen in human neurodegenerative diseases known to be sensitive to altered Cu homeostasis. b-amyloid is known to interact with Cu2+, increasing plaque burden and neurotoxicity. Increased Cu2+ is expected if Cu2+ is not converted to Cu+. To assess the impact of swip-10/MBLAC1 on b-amyloid plaques, studies with the GMC101 worm line were implemented. The GMC101 line expresses plaque forming human b-amyloid (1-42) peptide in muscle cells. The GMC101; swip-10 line expresses b-amyloid (1-42) in the context of a deleted swip-10 gene, modeling therefore the case where b-amyloid plaque forming processes in humans could be accelerated in the case of reduced MBLAC1 gene expression, and thereby increasing risk for AD-CVD. Worms were stained with a fluorescent Congo-red derivative to allow for visualization and quantitation of b-amyloid plaques. Data were analyzed by a one-way ANOVA with post-hoc tests of significance at different ages of the worms, demonstrating significantly greater plaque deposition in the context of a swip-10 mutation. (FIG. 9) The worm studies revealed a broad metabolic insult arising from loss of swip-10. To gain insights as to whether metabolic effects are also seen systemically in Mblac1 KO mice versus WT, an analysis of serum biomarkers typically assayed was performed to interrogate changes in organ metabolic function in mice and humans (FIG. 10). Blood serum samples were assayed commercially to evaluate biomarkers as follows: BUN=blood urea nitrogen, a measure of kidney function; cholesterol, produced systemically, predominantly by the liver; glucose, evaluated to examine liver and pancreatic function and sugar uptake/storage, as altered in metabolic syndromes; ALT=alanine transaminase, a measure of liver disease; CPK=creatine phosphokinase, a measure used to assess muscle and cardiac dysfunction; creatinine, a measure of kidney function; CRP=C-reactive protein, a measure of systemic inflammation that can be elevated in metabolic or infectious disease; Troponin I, a measure of cardiac muscle damage; Troponin II, a measure of cardiac muscle damage. Findings show a significant elevation in serum glucose, decreases in cholesterol levels, and elevated ALT and CPK levels, and reduced creatinine, suggesting a metabolic syndrome with likely involvement of liver, kidney and muscle.

Based on the data described herein, besides the use of SWIP-10/MBLAC1 genetic modifications in worms and mice to perform screens for novel therapeutic agents, purified SWIP-10/MBLAC1 protein, SWIP-10/MBLAC1 expressing cells, or extracts from SWIP-10/MBLAC1-expressing cells can be used to screen for agents that treat Cu homeostatic disorders which display diminished mitochondrial respiration and/or oxidative stress linked to Cu dyshomeostasis. Also, targeting of genes and molecules that regulate SWIP-10 or MBLAC1 expression or function in cell/animal models established to study these elements are of therapeutic value.

### Example 3—Methods and Assays for Screening for Agents that Normalize Traits Linked to Cu Dyshomeostasis Using Cultured Cells Derived from swip-10 Mutant Worms or Mblac1 Mutant Mice

Cultured cells derived from the swip-10 and MBLAC1 animals (e.g., mice, worms) described herein are used for screening for agents that normalize traits linked to Cu dyshomeostasis. Such assays can identify agents that modulate, e.g., halt, changes in Cu+ or Cu+ dependent molecules, enzymes or molecular pathways. The screening assays involve evaluation of Cu-dependent changes in vitro, where agents were applied to the cells to restore the changes back to wildtype levels. In a typical assay, cells from swip-10 KO or KD worms or Mblac1 KO or KD mice are cultured, the cultured cells are treated with (contacted with, subjected to) one or more test agents, and the treated cells are evaluated for normalization of Cu+ and/or one or more measures or markers that is indicative of normalization of Cu homeostasis. Therapeutic and/or test agents can be added for a specific time period to allow for agent penetration into cells and to induce changes in, as examples, enzyme activities, mitochondrial function, histone production, and mRNAs, etc. In the experiments described above these various measures were shown to be changed in worms and mice. Test agents can also be added repeatedly and in the presence of drugs that block Cu related pathways (e.g. Cu chelators) to thereby test the Cu dependence of changes observed with loss of swip-10 and Mblac1.

The cells can be primary cell cultures used for short term studies or the cells can be transformed with a virus to immortalize them for long term use (i.e., immortalized cells). Stem cells, isolated from the mice, that can differentiate into cells forming different organs can be used so as to model changes in these tissues. The cells can be grown as monolayers on culture plates or in suspension for non-adherent cells (e.g, immune cells), or they could be grown as mixed cultures, with cultures made up of specific combinations of different cells, or they could be grown as cell aggregates with the ability to generate 3D structures (e.g., as organoids). Any other suitable cells from the animals can be used, including mixed cultures of neurons and glia, glial cells, neuronal cells, hepatocytes, cardiac cells, differentiatable embryonic stem(ES) cells, and muscle cells.

### Other Embodiments

Any improvement may be made in part or all of the method steps, assays, reagents, and compositions. All references, including publications, patent applications, and patents, cited herein are hereby incorporated by reference in their entireties. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. A method to identify agents or manipulations that modulate copper (Cu) dyshomeostasis in an Mblac1- or swip-10-dependent manner comprising:
(i) providing a plurality of Mblac1 or swip-10 knockdown (KD) or knockout (KO) animals, and a plurality of Mblac1 or swip-10 wild-type (WT) animals;
(ii) exposing a first portion of the plurality of Mblac1 or swip-10 KD or KO animals and a first portion of the plurality of the Mblac1 or swip-10 WT animals to at least one test agent or manipulation, and exposing a second portion of the plurality of Mblac1 or swip-10 KD or KO animals and a second portion of the Mblac1 or swip-10 WT animals to a control vehicle;
(iii) collecting a sample from each animal;
(iv) measuring a value for at least one Cu marker in the samples from all the animals and comparing the effects of the at least one test agent or manipulation between the first portion of the plurality of Mblac1 or swip-10 KD or KO animals and the second portion of the plurality of the Mblac1 or swip-10 WT animals; and
(v) identifying the at least one test agent or manipulation as one that modulates Cu dyshomeostasis if any of the following are observed: the value for the at least one Cu marker from the first portion of the plurality of Mblac1 or swip-10 KD or KO animals is statistically different from the value for the at least one Cu marker from the second portion of the plurality of Mblac1 or swip-10 KD or KO animals; the value for the at least one Cu marker from the first portion of the plurality of Mblac1 or swip-10 WT animals is statistically different from the value for the at least one Cu marker from the second portion of the plurality of Mblac1 or swip-10 WT animals; and the value for the at least one Cu marker from the first portion of the plurality of Mblac1 or swip-10 KD or KO animals is equal or similar to the value for the at least one Cu marker from the first and/or second portions of the plurality of the Mblac1 or swip-10 WT animals.

2. The method of claim 1, wherein modulating Cu dyshomeostasis comprises alleviating Cu dyshomeostasis such that Cu homeostasis is normalized; wherein the at least one Cu marker is selected from the group consisting of: one or more Cu redux states, a Cu-dependent enzyme, a Cu-binding protein, a Cu-dependent physiological response or behavior, a Cu-dependent process, and a change in RNA, protein, posttranslational protein modification, or metabolite linked to Cu homeostasis; and wherein in step iv), the effects of the at least one test agent or manipulation comprise a change relative to pre-exposure in at least one of: Cu redux states, a Cu-dependent enzyme, a Cu-binding protein, a Cu-dependent physiological response or behavior, a Cu-dependent process, and a change in RNA, protein, posttranslational protein modification, or metabolite linked to Cu homeostasis.

3. (canceled)

4. (canceled)

5. The method of claim 1, wherein modulating Cu dyshomeostasis comprises increasing Cu dyshomeostasis.

6. The method of claim 1, wherein the at least one test agent is selected from the group consisting of: small molecule, drug, nucleic acid, protein, peptide, nanoparticle, virus, viral vector, Cu chelator in a swip-10/Mblac1-dependent manner, Cu chaperone in a swip-10/MBLAC1-dependent manner, enzyme that binds Cu, protein that removes Cu, Cu transporter, agent that modifies enzymatic activity of Cu in a swip-10/Mblac1-dependent manner, and histone enzymatic modifying agent that acts in a swip-10/Mblac1-dependent manner; and wherein the at least one manipulation is selected from the group consisting of: a genetic manipulation; a transcriptome manipulation, a metabolome manipulation, and an environmental manipulation linked to Cu dyshomeostasis.

7. (canceled)

8. The method of claim 1, wherein in step iii) the sample is brain tissue, a peripheral tissue, a bodily fluid, or feces; and wherein in the plurality of MBLAC1 or swip-10 KD or KO animals, Mblac1 expression is knocked down or knocked out selectively in glial cells.

9. (canceled)

10. A method to identify agents or manipulations that modulate Cu dyshomeostasis in an Mblac1- or swip-10-dependent manner comprising:
(i) providing a plurality of Mblac1 or swip-10 KD or KO animals, and a plurality of Mblac1 or swip-10 WT animals;
(ii) measuring a baseline value for at least one Cu marker for each animal;
(iii) exposing each animal to at least one test agent or manipulation, wherein each animal provides its own baseline value prior to exposure;
(iv) at a first post-exposure time point, measuring at least one exposure response value for the at least one Cu marker for each animal;
(v) optionally, at one or more of a second, third, fourth, fifth and sixth post-exposure time point, measuring an exposure response value for the at least one Cu marker for each animal;
(vi) averaging the WT exposure response values compared to the WT baseline values, averaging the KO/KD exposure response values compared to the KO/KD baseline values, and then comparing the WT average to the KO or KD average, wherein if the WT average is different relative to the KO or KD average, the at least one test agent or manipulation modulates Cu dyshomeostasis.

11. The method of claim 10, wherein step (v) is performed and a time course of the exposure response to the at least one test agent or manipulation is determined; wherein the at least one test agent is detectably labeled; and wherein in step (ii), measuring a baseline value for at least one Cu marker comprises collecting a sample from the animals, in vivo imaging of a tissue in the animals, or measuring physiological behavior of all animals.

12. (canceled)

13. (canceled)

14. The method of claim 10, wherein in step (iv), measuring at least one exposure response value for the at least one Cu marker comprises collecting a sample from the animals, in vivo imaging of a tissue in the animals, or measuring physiological behavior of all animals; and wherein in step (ii) measuring a baseline value for at least one Cu marker and in step (iv) measuring at least one exposure response value for the at least one Cu marker, blood from the animals is analyzed and the at least one Cu marker is measured and Cu-dependent behavior in the animals is analyzed and measured.

15. (canceled)

16. The method of claim 11, wherein the samples comprise blood, serum, saliva, urine or feces.

17. The method of claim 10, wherein modulating Cu dyshomeostasis comprises alleviating Cu dyshomeostasis such that Cu homeostasis is normalized in the subject.

18. The method of claim 10, wherein the at least one Cu marker is selected from the group consisting of: one or more Cu redox states, a Cu-dependent enzyme, a Cu-binding protein, a Cu-dependent physiological response or behavior, a Cu-dependent process, and a change in RNA, protein, posttranslational protein modification, or metabolite linked to Cu homeostasis.

19. The method of claim 10, wherein modulating Cu dyshomeostasis comprises increasing Cu dyshomeostasis.

20. The method of claim 10, wherein the at least one test agent is selected from the group consisting of: small molecule, drug, nucleic acid, protein, peptide, nanoparticle, virus, viral vector, Cu chelator in a swip-10/Mblac1-dependent manner, Cu chaperone in a swip-10/MBLAC1-dependent manner, enzyme that binds Cu, protein that removes Cu, Cu transporter, agent that modifies enzymatic activity of Cu in a swip-10/Mblac1-dependent manner, and histone enzymatic modifying agent that acts in a swip-10/Mblac1-dependent manner; and wherein the at least one manipulation is selected from the group consisting of: a genetic manipulation; a transcriptome manipulation, a metabolome manipulation, and an environmental manipulation linked to Cu dyshomeostasis.

21. (canceled)

22. The method of claim 10, wherein in the plurality of MBLAC1 or swip-10 KD or KO animals, Mblac1 expression is knocked down or knocked out selectively in glial cells.

23. A method to identify agents that support Cu-dependent neuronal health in a subject comprising:
(i) providing a plurality of Mblac1 or swip-10 KD or KO animals, and a plurality of MBLAC1 or swip-10 WT animals;
(ii) exposing a first portion of the plurality of Mblac1 or swip-10 KD or KO animals and a first portion of the plurality of Mblac1 or swip-10 WT animals to a neural toxin or other neural insult that results in pathology in a Cu-dependent manner, wherein a second portion of the Mblac1 or swip-10 KD or KO animals and a second portion of the Mblac1 or swip-10 WT animals are not exposed to the neural toxin or neural insult as controls;
(iii) measuring the pathology in each exposed animal;
(iv) administering a test agent to at least the first portion of the plurality of Mblac1 or swip-10 KD or KO animals and the first portion of the plurality of Mblac1 or swip-10 WT animals;
(v) measuring the pathology in each animal that was administered the test agent;
(vi) identifying the test agent as an agent that supports Cu-dependent neuronal health in a subject if the test agent reverses the pathology of an Mblac1 KO.

24. The method of claim 23, wherein measuring the pathology in the animals comprises measuring visually, biochemically, physiologically or behaviorally a marker of neuronal damage in the animals; wherein the identified agent supports Cu-dependent neuronal health in the presence of MBLAC1 but not in the absence of MBLAC1; wherein in step (ii), the neural toxin or other neural insult induces oxidative stress and/or neural degeneration in the animals; and wherein the identified agent has neuroprotective activity when administered to a mammalian subject in need thereof.

25. (canceled)

26. (canceled)

27. (canceled)

28. The method of claim 24, wherein the plurality of Mblac1 or swip-10 KD or KO animals are swip-10 KD or KO C. elegans animals, and the plurality of Mblac1 or swip-10 WT animals are swip-10 WT. C. elegans animals.

29. The method of claim 24, wherein pathology is measured by measuring proteins that are stimulated during cell death progression, by measuring the number of neural cells, or by an anatomical assay that counts healthy parts of a cell including dendrites or cell bodies; and wherein in the plurality of MBLAC1 or swip-10 KD or KO animals, Mblac1 expression is knocked down or knocked out selectively in glial cells.

30. (canceled)

31. A method to identify agents or manipulations that modulate Cu dyshomeostasis in an Mblac1- or swip-10-dependent manner comprising:
(i) providing a first group of cultured cells derived from a plurality of Mblac1 or swip-10 KD or KO animals, and a second group of cultured cells derived from a plurality of Mblac1 or swip-10 WT animals;
(ii) exposing a first portion of the first group of cultured cells and a first portion of the second group of cultured cells to at least one test agent or manipulation, and exposing a second portion of the first group of cultured cells and a second portion of the second group of cultured cells to a control vehicle;
(iii) measuring a value for at least one Cu marker in each of the portions and comparing the effects of the at least one test agent or manipulation between the first portion of the first group of cultured cells and the second portion of the second group of cultured cells; and
(iv) identifying the at least one test agent or manipulation as one that modulates Cu dyshomeostasis if at least one of the following is observed: the value for the at least one Cu marker from the first portion of the first group of cultured cells is statistically different from the value for the at least one Cu marker from the second portion of the first group of cultured cells; the value for the at least one Cu marker from the first portion of the second group of cultured cells is statistically different from the value for the at least one Cu marker from the second portion of the second group of cultured cells; and the value for the at least one Cu marker from the first portion of the first group of cultured cells animals is equal or similar to the value for the at least one Cu marker from the first and/or second portions of the second group of cultured cells.

32. A method to identify agents or manipulations that modulate Cu dyshomeostasis in an Mblac1- or swip-10-dependent manner comprising:
(i) providing a first group of cultured cells derived from a plurality of Mblac1 or swip-10 KD or KO animals, and a second group of cultured cells derived from a plurality of Mblac1 or swip-10 WT animals;
(ii) measuring a baseline value for at least one Cu marker in both groups of cultured cells;
(iii) exposing both groups of cultured cells to at least one test agent or manipulation, wherein each cultured cell provides its own baseline value prior to exposure;
(iv) at a first post-exposure time point, measuring at least one exposure response value for the at least one Cu marker for both groups of cultured cells;
(v) optionally, at one or more of a second, third, fourth, fifth and sixth post-exposure time point, measuring an exposure response value for the at least one Cu marker for both groups of cultured cells; and
(vi) averaging the second group of cultured cells' exposure response values compared to the second group of cultured cells' baseline values, averaging the first group of cultured cells' exposure response values compared to the first group of cultured cells' baseline values, and then comparing the second group of cultured cells' average to the first group of cultured cells' average, wherein if the second group of cultured cells' average is different relative to the first group of cultured cells' average, the at least one test agent or manipulation modulates Cu dyshomeostasis.
